(19) United States
(12) Patent Application Publication
(10) Pub. No.: US 2026/0144990 A1
(43) Pub. Date: May 28, 2026

(54) DETERMINING STIMULUS DELIVERY FOR SENSING BIOELECTRICAL SIGNALS

(71) Applicant: Medtronic, Inc., Minneapolis, MN (US)

(72) Inventors: Lucien Talvard, Paris (FR); Andrew J. Cleland, Macquarie Park (AU)

(21) Appl. No.: 19/377,519

(22) Filed: Nov. 3, 2025

Related U.S. Application Data
(60) Provisional application No. 63/724,094, filed on Nov. 22, 2024.

(51) Int. Cl. A61N 1/36

(52) U.S. Cl. CPC A61N 1/36139; A61N 1/36062; A61N 1/36171

(57) ABSTRACT

Devices, systems, and techniques include determining stimulation parameters to facilitate sensing bioelectric signals. In one example, a system includes processing circuitry configured to control stimulation generation circuitry to deliver at least one first pulse from a first electrode combination and control sensing circuitry to sense an electrical signal during a sensing window associated with delivery of a second pulse from a second electrode combination different than the first electrode combination. The processing circuitry can then generate an analysis of the electrical signal sensed during the sensing window, the analysis configured to identify one or more artifacts associated with the at least one first stimulus from the first electrode combination and determine, based on the analysis, a stimulus schedule for a plurality of subsequent pulses deliverable from the first electrode combination.

### CROSS-REFERENCE TO RELATED APPLICATION

This application claims the benefit of and priority to U.S. Provisional Patent Application No. 63/724,094 filed Nov. 22, 2024, the entire disclosure of which is incorporated by reference herein.

### TECHNICAL FIELD

This disclosure generally relates to stimulation therapy, and more specifically, sensing bioelectrical signals for controlling electrical stimulation therapy.

### BACKGROUND

Medical devices may be external or implanted and may be used to deliver electrical stimulation therapy to patients via various tissue sites to treat a variety of symptoms or conditions such as chronic pain, dysautonomia, motor deficit, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as “spinal cord stimulation” (SCS), “sacral neuromodulation” (SNM), “deep-brain stimulation” (DBS), and “peripheral-nerve stimulation” (PNS), respectively.

Electrical stimulation therapy may be delivered by the medical device in a “train” of electrical stimulation pulses, and parameters that define the electrical stimulation pulses may include a frequency, an amplitude, a pulse width, and a pulse shape.

### SUMMARY

Systems, devices, and techniques are described for configuring and controlling electrical stimulation therapy for a patient. In particular, the techniques described herein include analyzing sensed signals from one or more electrode combinations and determining a stimulus schedule for electrical stimulation based on the analysis. In some examples, systems may sense electrical signals in a patient and use those sensed signals as feedback for adjusting stimulation over time. Evoked signals are example electrical signals that are elicited by a delivered stimulus, and one example of evoked signals includes evoked compound action potential (ECAP) signals.

The system can determine a stimulus schedule from one or more electrode combinations in order to enable sensing of evoked signals, such as ECAP signals, and reduce “artifacts” in the sensed signal. The artifacts may be representations of the delivered stimulus and/or unwanted ECAP signals within the target sensed evoked signals. For example, the system can deliver different stimulation pulses that may have different amplitudes, frequencies, pulse widths, etc. In some examples, these different stimulation pulses may be delivered as different pulse trains. In some examples the different stimulation pulses may be delivered from electrode combinations using different leads and/or sensing electrode combinations on different leads from the stimulation electrode combinations from which stimulation pulses are delivered. The system may be configured to detect an evoked signal, such as an ECAP signal, that is evoked from one target pulse (or pulses) instead of other pulses. In order to capture the desired ECAP signal and avoid artifacts from other delivered pulses, the system can sense an electrical signal during a sensing window associated with the target pulse and analyze the electrical signal for any artifacts and undesired ECAPs. The system can then adjust the stimulus schedule for the other subsequent pulses in order to reduce or eliminate artifacts or unwanted ECAPs from sensed electrical signals.

In one example, a system includes processing circuitry configured to control stimulation generation circuitry to deliver at least one first pulse from a first electrode combination; control sensing circuitry to sense an electrical signal during a sensing window associated with delivery of a second pulse from a second electrode combination different than the first electrode combination; generate an analysis of the electrical signal sensed during the sensing window, the analysis configured to identify one or more artifacts associated with the at least one first stimulus from the first electrode combination; and determine, based on the analysis, a stimulus schedule for a plurality of subsequent pulses deliverable from the first electrode combination.

In another example, a method includes controlling, by processing circuitry, stimulation generation circuitry to deliver at least one first pulse from a first electrode combination; controlling, by the processing circuitry, sensing circuitry to sense an electrical signal during a sensing window associated with delivery of a second pulse from a second electrode combination different than the first electrode combination; generating, by the processing circuitry, an analysis of the electrical signal sensed during the sensing window, the analysis configured to identify one or more artifacts associated with the at least one first stimulus from the first electrode combination; and determining, by the processing circuitry and based on the analysis, a stimulus schedule for a plurality of subsequent pulses deliverable from the first electrode combination.

In another example, a non-transitory computer-readable storage medium comprising instructions that, when executed, causes processing circuitry to control stimulation generation circuitry to deliver at least one first pulse from a first electrode combination; control sensing circuitry to sense an electrical signal during a sensing window associated with delivery of a second pulse from a second electrode combination different than the first electrode combination; generate an analysis of the electrical signal sensed during the sensing window, the analysis configured to identify one or more artifacts associated with the at least one first stimulus from the first electrode combination; and determine, based on the analysis, a stimulus schedule for a plurality of subsequent pulses deliverable from the first electrode combination.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes a medical device programmer and an implantable medical device (IMD) configured to deliver spinal cord stimulation (SCS) therapy to a patient with a spinal cord injury (SCI).

FIG. 2A is a block diagram illustrating some example components of the IMD of FIG. 1.

FIG. 2B is a block diagram of some example components of the external programmer of FIG. 1.

FIG. 3 is a graph of an example of evoked compound action potentials (ECAPs) sensed for respective stimulation pulses.

FIG. 4A is a timing diagram illustrating an example of electrical stimulation pulses and respective sensed ECAPs.

FIG. 4B is a timing diagram illustrating another example of electrical stimulation pulses and respective sensed ECAPs.

FIGS. 5A and 5B are conceptual diagrams of example leads and electrodes for delivering electrical stimulation and sensing ECAP signals.

FIG. 6 is a timing diagram illustrating example blanking of pulses to generate a schedule for stimulation pulses.

FIG. 7 is a flowchart illustrating an example technique for determining an stimulus schedule based on sensed signals from an electrode combination.

FIG. 8 is a flowchart illustrating an example technique for adding and removing pulses based on identified artifacts to determine a stimulus schedule for stimulation.

FIG. 9 is a flowchart illustrating an example technique for determining electrode migration based on a change in identified artifacts compared to a prior period of time.

FIG. 10 is a flowchart illustrating an example technique for selecting a sense electrode combination and determining a stimulus schedule for adjusting stimulation therapy.

### DETAILED DESCRIPTION

The disclosure describes examples of medical devices, systems, and techniques for configuring and controlling electrical stimulation therapy for a patient, such as controlling stimulation to also enable sensing of electrical signals from the patient. Electrical stimulation therapy is typically delivered to a target tissue (e.g., nerves of the spinal cord or muscle) of a patient via two or more electrodes. Parameters of the electrical stimulation therapy (e.g., electrode combination, voltage or current amplitude, pulse width, pulse frequency, etc.) are selected by a clinician and/or the patient to provide relief from various symptoms, such as pain, nervous system disorders, muscle disorders, etc. However, as the patient moves, the distance between the electrodes and the target tissues changes. Since neural recruitment at the nerves is a function of stimulation intensity (e.g., amplitude and/or pulse frequency) and distance between the target tissue and the electrodes, movement of the electrode closer to the target tissue may result in increased neural recruitment (e.g., possible painful sensations or adverse motor function), and movement of the electrode further from the target tissue may result in decreased efficacy of the therapy for the patient. Certain patient postures (which may or may not include patient activity) may be representative of respective distances (or changes in distance) between electrodes and nerves and thus be an informative feedback variable for modulating stimulation therapy.

Evoked signals are generally electrical signals or other signals that can be evoked within the patient from delivered electrical stimulation (e.g., one or more pulses). In some example, ECAPs are a measure of neural recruitment because each ECAP signal represents the superposition of electrical potentials generated from a population of axons firing in response to an electrical stimulus (e.g., a stimulation pulse). Changes in a characteristic (e.g., an amplitude of a portion of the signal or area under the curve of the signal) of an ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude). Therefore, a system can determine that the distance between electrodes and nerves has increased or decreased in response to determining that the measured ECAP characteristic value has increased or decreased. For example, if the set of parameter values stays the same and the ECAP characteristic value of amplitude increases, the system can determine that the distance between electrodes and the nerve has decreased. ECAPs may also be used to identify other changes within the patient and/or the tissue-electrode interface. Other examples of evoked signals may include evoked resonant neural activity (ERNA) signals. In some examples, other sensed signals may also be used, such as local field potential (LFP) signals.

In some examples, effective stimulation therapy may rely on a certain level of neural recruitment at a target nerve. This effective stimulation therapy may provide relief from one or more conditions (e.g., patient perceived pain) without an unacceptable level of side effects (e.g., overwhelming perception of stimulation). If the patient changes posture or otherwise engages in physical activity, the distance between the electrodes and the nerve changes as well. This change in distance can cause loss of effective therapy and/or side effects if the parameter values that define stimulation pulses are not adjusted to compensate for the change in distance. A system may change stimulation parameters to compensate for changes to the distance between electrodes and the target nerve, such as increasing stimulation intensity in response the distance increases and decreasing stimulation intensity in response to the distance decreasing.

Although the system may adjust one or more stimulation parameters according to the one or more characteristics of an evoked signal to compensate for the change in distance between electrodes and nerves, the system may have difficulty detecting evoked signals during certain situations in which noise is present in the sensed electrical signals. This noise can be due to activity in or around the patient, such as delivered electrical stimulation itself. For example, delivery of a stimulation pulse during a sensing window configured to detect an evoked signal can interfere with, or obscure, the detection of the evoked signal. The stimulation pulse is typically orders of magnitude greater in amplitude than the evoked signal and can obscure the evoked signal. This stimulation pulse can be a problem when trying to detect evoked signals if a system is delivering a train of stimulation pulses, or multiple different trains of stimulation pulses from different electrode combinations, that may overlap in time with evoked signals that the system is attempting to detect. Timing the delivery of stimulation pulses may be more straightforward for sensing electrodes at a known distance from stimulation electrodes, but stimulation electrodes on different leads or other structures from the sensing electrodes can further complicate the scheduling of stimulation pulses around sensing windows needed to sense and detect evoked signals.

As described herein, a medical device may be configured to determine a schedule for delivering electrical stimulation that reduces artifacts from obscuring evoked signals. The system can be configured to deliver one or more stimulation pulses, sense electrical signals, and analyze the sensed electrical signals for any artifacts that may be present within the electrical signal. Based on the timing of the artifacts within the electrical signals, the system can schedule subsequent stimulation pulses in order to reduce or prevent future artifacts from subsequent sensing windows. In some examples, scheduling the subsequent stimulation pulses may include moving, removing, or even adding one or more stimulation pulses from a planned train of stimulation pulses. This scheduling of stimulation pulses may compensate for unknown distances between stimulation electrodes and sensing electrodes and transient times for the stimulation pulses or other signals (e.g., non-targeted ECAP signals or other evoked signals) to be detected by the sensing electrodes of the system. In some examples, the initial schedule may be predetermined, based on historical data, or even based on patient imaging (e.g., x-ray or fluoroscopy imaging) of electrodes. For example, the system may measure distances between electrodes in the images and estimate timing of stimulation based on the distances and estimated conduction velocities (e.g., average tissue conduction velocity or even tissue-specific conduction velocities).

By scheduling stimulation pulse to enable detection of evoked signals, a system may then sense, via a same or different electrode combination, an evoked signal such as an ECAP signal elicited by a stimulation pulse (e.g., a control stimulation pulse). The system may identify at least one characteristic (e.g., may measure at least one ECAP parameter value) of the sensed ECAP signal. Various non-limiting examples of identifiable characteristics of ECAP signals include a signal amplitude, a signal width, a signal latency (e.g., a duration between delivery of the control pulse and sensing of the ECAP signal), a signal slope, an area-under-the-curve (AUC) of an ECAP signal, a signal phasing, a signal curvature, an oscillatory signal pattern, a signal morphology; or a temporal stability or temporal variance of an ECAP signal.

In response to identifying at least one ECAP characteristic, the system may determine a therapy parameter value that at least partially defines an “informed” or “therapeutic” stimulation pulse or pulses configured to treat a patient condition. As used herein, a “therapy” or “treatment” of a patient condition may reduce or prevent symptoms such as pain in one or more limbs, back, or other area of the patient, but other symptoms or benefits may result from other therapies as well. For instance, the informed stimulation pulses may be configured to reduce pain symptoms for a patient, either by masking pain sensation via paresthesia and/or reducing the pain signals themselves. These examples are merely illustrative, and the techniques described herein may be applied similarly for the treatment of any condition.

In some examples, the control stimulation pulses may be defined by one or more stimulation parameters, other than just amplitude, different than the informed stimulation pulses. For example, the control stimulation pulses may be defined by a different pulse width, frequency, electrode combination, etc. These different stimulation parameters of the control stimulation pulses may enable the system to elicit detectable ECAP signals that can be used to adjust the informed stimulation pulses configured to provide therapy to the patient. In situations where the control stimulation pulses themselves can contribute to therapy, the system may only deliver control stimulation pulses and adjust a parameter, such as amplitude, of subsequent control stimulation pulses based on the ECAP signal elicited by a prior control stimulation pulse. In some examples, a system may deliver one or more informed pulses between respective control pulses in order to provide therapy and sense ECAP signals during the therapy regime. In this manner, a train of informed pulses may be at least partially interleaved with a train of control pulses. The informed and control pulses may alternate every pulse on a 1:1 bases, multiple informed pulses may be delivered between each control pulse, or multiple control pulses may be delivered between each informed pulse. The system may adjust the ratio of informed pulses to control pulses over time as needed to maintain therapy and/or sense ECAP signals at a sufficient frequency to provide feedback for the informed pulses. In some examples, informed stimulation pulses and control stimulation pulses may elicit evoked signals. However, the system may be configured to detect evoked signals elicited by one or more control stimulation pulses instead of any informed stimulation pulses.

In some examples, the system may be configured to “sample” (e.g., sense) an ECAP signal according to a predetermined sampling frequency, which may correspond to selected stimulation pulses that elicit an ECAP or a frequency of control stimulation pulses. As one non-limiting example, the predetermined sampling frequency may be from about 1 Hz to about 100 Hz, or about 50 Hz in one example.

In some examples, but not all examples, in the control stimulation pulse may be configured to at least partially contribute to the therapy (e.g., elicit a therapeutic response) provided to the patient, e.g., in addition to eliciting the ECAP signal. In other examples, the control stimulation pulse may only be configured to elicit the ECAP signal, e.g., without contributing to a therapeutic effect for the patient.

Although SCS is generally described herein in the form of electrical stimulation “pulses,” SCS may be delivered in non-pulse form in other examples. For example, SCS may be delivered as a signal having various waveform shapes, frequencies, and amplitudes. Therefore, electrical stimulation in the form of a non-pulse signal may be a continuous signal than may have a sinusoidal waveform or other continuous waveform. SCS therapy is generally described herein as one example. However, similar techniques may be used to provide other therapies of the same or other anatomical regions of the patient, such as pelvic floor stimulation, tibial stimulation, sacral nerve stimulation, peripheral nerve stimulation, deep brain stimulation, etc. Moreover, ECAP signals are generally described as an example sensed signal that can be used for adjusting a stimulus schedule. However, other signals such as evoked resonant neural activity (ERNA) signals or other sensed signals, such as local field potential (LFP) signals, may be used instead of or in addition to ECAP signals.

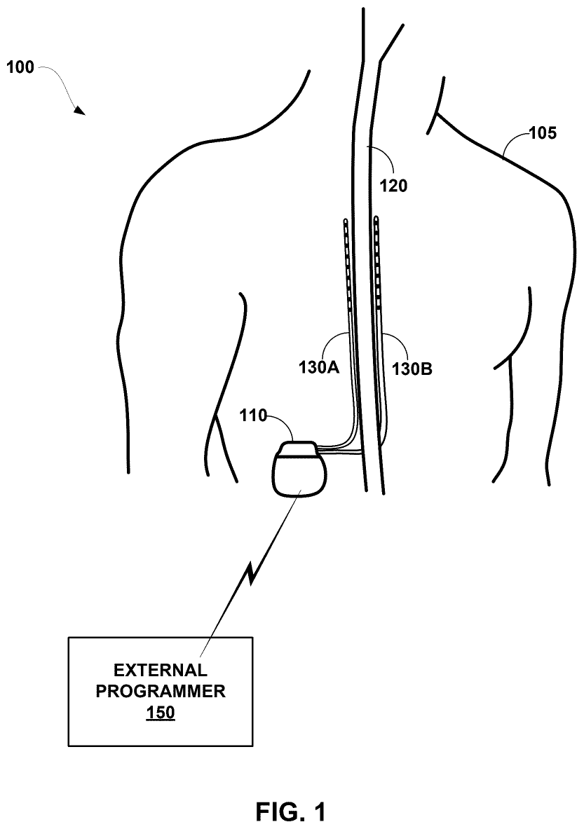

FIG. 1 is a conceptual diagram illustrating an example spinal-cord-stimulation (SCS) system 100 that includes implantable medical device (IMD) 110 configured to deliver electrical stimulation therapy, and in particular, SCS therapy, to a patient 105 afflicted with various symptoms such as pain symptoms. Although the techniques described in this disclosure are generally applicable to a variety of medical devices including external (e.g., epidural) and implantable medical devices (IMDs), application of such techniques to IMDs and, more particularly, implantable electrical stimulators (e.g., neurostimulators) will be described for purposes of illustration.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes of electrodes of leads 130A and/or 130B (collectively, “leads 130,” or in the alternative, “lead 130”), e.g., for treatment of one or more conditions resulting directly from, or otherwise associated with, the patient's SCI 140. In other examples, IMD 110 may be coupled to a single lead 130 carrying multiple electrodes or more than two leads each carrying multiple electrodes. In addition to electrical stimulation therapy, IMD 110 may also be configured to generate and deliver “control” stimulation pulses configured to elicit ECAP signals that may or may not contribute to the therapy provided by “informed” stimulation pulses. As discussed herein, the control pulses may be non-therapeutic in some examples. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In other examples, leads 130 are positioned externally to the patient's epidermis. In some examples, IMD 110 uses one or more leads 130, while in other examples, IMD 110 is leadless.

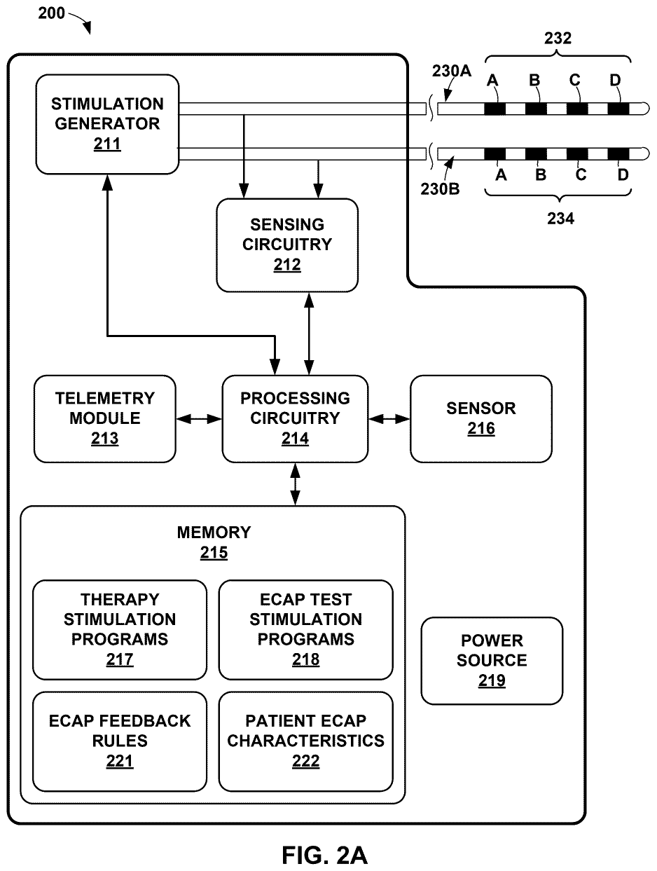

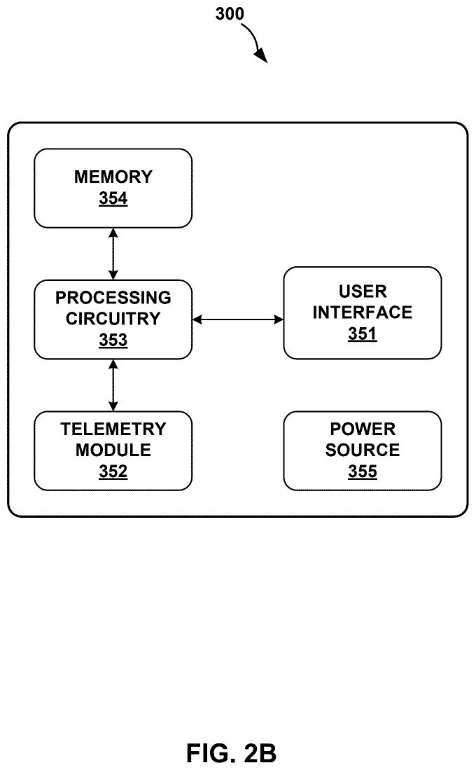

IMD 110 may be constructed of any polymer, metal, or composite material sufficient to house the components of IMD 110 (e.g., components illustrated in FIG. 2A) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks, or other suitable site within patient 105. The outer housing of IMD 110 may be configured to provide a hermetic seal for components, such as a rechargeable or non-rechargeable power source. In addition, in some examples, the outer housing of IMD 110 may be selected from a material that facilitates receiving energy to charge the rechargeable power source.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator in IMD 110 to tissue of patient 105.

Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead 130 or more than two leads 130, each coupled to IMD 110 and directed to similar or different target tissue sites.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays may include electrode segments, which may be arranged at respective positions around a periphery of a lead, e.g., arranged in the form of one or more segmented rings around a circumference of a cylindrical lead. In other examples, one or more of leads 130 are linear leads having 8 ring electrodes along the axial length of the lead. In another example, the electrodes are segmented rings arranged in a linear fashion along the axial length of the lead and at the periphery of the lead.

The stimulation parameter of a therapy stimulation program that defines the stimulation pulses of electrical stimulation therapy by IMD 110 through the electrodes of leads 130 may include information identifying which electrodes have been selected for delivery of stimulation according to a stimulation program, the polarities of the selected electrodes, i.e., the electrode combination for the program, and voltage or current amplitude, pulse frequency, pulse width, or pulse shape of stimulation delivered by the electrodes. These stimulation parameters of informed pulses are typically predetermined parameter values determined prior to delivery of the informed pulses. However, in some examples, system 100 may change one or more parameter values automatically based on one or more factors or based on user input.

In addition to stimulation informed pulses, an ECAP “test” stimulation program may define stimulation parameter values that define control pulses delivered by IMD 110 through at least some of the electrodes of leads 130. These stimulation parameter values may include information identifying which electrodes have been selected for delivery of control pulses, the polarities of the selected electrodes, i.e., the electrode combination for the program, and voltage or current amplitude, pulse frequency, pulse width, and pulse shape of stimulation delivered by the electrodes. The stimulation signals (e.g., one or more stimulation pulses or a continuous stimulation waveform) defined by the parameters of each ECAP test stimulation program are configured to evoke a compound action potential from nerves. In some examples, the ECAP test stimulation program may define when the control pulses are to be delivered to the patient based on the frequency and/or pulse width of the informed pulses. However, the stimulation defined by each ECAP test stimulation program are not intended to provide or contribute to therapy for the patient. In an example where the control pulses contribute to or provide therapy for the patient, the ECAP test stimulation program may also be used in place of, or be the same as, a therapy stimulation program.

In some examples, IMD 110 (e.g., lead 130) may include one or more sensors configured to allow IMD 110 to monitor one or more parameters of patient 105, such as patient activity, pressure, temperature, movement, or other characteristics. As non-limiting examples, sensors of IMD 110 may include accelerometer(s), EEG(s), EMG(s), or the like. The one or more sensors may be provided in addition to, or in place of, therapy delivery by lead 130.

IMD 110 is configured to deliver SCS therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions.

In other examples, stimulation of spinal cord 120 may prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

IMD 110 generates and delivers electrical stimulation therapy to a target stimulation site within patient 105 via the electrodes of leads 130 to patient 105 according to one or more therapy stimulation programs. A therapy stimulation program defines values for one or more parameters that define an aspect of the therapy delivered by IMD 110 according to that program. For example, a therapy stimulation program that controls delivery of stimulation by IMD 110 in the form of pulses may define values for voltage or current pulse amplitude, pulse width, and pulse rate (e.g., pulse frequency) for stimulation pulses delivered by IMD 110 according to that program. A therapy stimulation program may define control pulses and/or informed pulses when these pulses are configured to contribute to the therapeutic effect (e.g., paresthesia, pain blocking, etc.) for the patient.

Furthermore, IMD 110 is configured to deliver control stimulation to patient 105 via a combination of electrodes of leads 130, alone or in combination with an electrode carried by or defined by an outer housing of IMD 110. The tissue targeted by the control stimulation may be the same tissue targeted by the electrical stimulation therapy, but IMD 110 may deliver control pulses via the same, at least some of the same, or different electrodes, and intended to elicit a detectable evoked signal, such as an ECAP signal. This control stimulation may (e.g., therapeutic stimulation) or may not (e.g., non-therapeutic stimulation) contribute to a therapeutic effect for the patient. Since control pulses can be delivered in an interleaved manner with informed pulses, a clinician and/or user may select any desired electrode combination for informed pulses. Like the electrical stimulation therapy, the control stimulation may be in the form of electrical stimulation pulses or continuous waveforms.

In one example, each control pulse may include a balanced, bi-phasic square pulse that employs an active recharge phase. However, in other examples, the control pulses may include a monophasic pulse followed by a passive recharge phase. In other examples, a control pulse may include an imbalanced bi-phasic portion and a passive recharge portion. Although not necessary, a bi-phasic control pulse may include an interphase interval between the positive and negative phase to promote propagation of the nerve impulse in response to the first phase of the bi-phasic pulse. The control stimulation may be delivered without interrupting the delivery of the electrical stimulation informed pulses, such as during the window between consecutive informed pulses. The control pulses may elicit an ECAP signal from the tissue, and IMD 110 may sense the ECAP signal via two or more electrodes on leads 130. In cases where the control pulses are applied to spinal cord 120, the signal may be sensed by IMD 110 from spinal cord 120. As discussed herein, the control stimulation may contribute, alone or in part, to the therapeutic effect received by the patient. In other words, control pulses may be delivered to provide therapy without any additional informed pulses in some examples. In examples in which the control pulses alone can provide therapy to the patient, the control stimulation may be the therapy stimulation for that patient. IMD 110 may be configured to schedule delivery of stimulation pulses (e.g., control stimulation pulses and informed stimulation pulses) in order to create a sensing window that is timed to reduce potential artifacts from occurring over the ECAP signals intended for detection.

In some examples, but not all examples, IMD 110 may deliver control stimulation to a target stimulation site within patient 105 via the electrodes of leads 130 according to one or more ECAP test stimulation programs. The one or more ECAP test stimulation programs may be stored in a memory of IMD 110. Each ECAP test program of the one or more ECAP test stimulation programs includes values for one or more parameters that define an aspect of the control stimulation delivered by IMD 110 according to that program, such as current or voltage amplitude, pulse width, pulse frequency, electrode combination, and, in some examples, timing based on informed pulses to be delivered to patient 105. In some examples, IMD 110 delivers control stimulation to patient 105 according to multiple ECAP test stimulation programs.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from programmer 150 to control electrical stimulation therapy (e.g., informed pulses, and in some examples control pulses) and control stimulation (e.g., control pulses). For example, external programmer 150 may transmit therapy stimulation programs, ECAP test stimulation programs, stimulation parameter adjustments, therapy stimulation program selections, ECAP test program selections, user input, or other information to control the operation of IMD 110, e.g., by wireless telemetry or wired connection. In some examples, IMD 110 and/or programmer 150 may be configured to modify one or more therapy stimulation programs, ECAP test stimulation programs, or any other instructions in order to schedule stimulation pulses to reduce artifacts from a sensing window or otherwise interfering with expected ECAP signals from being detected.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and programmer 150 may communicate via wireless communication using any techniques known in the art. Examples of communication techniques may include, for example, radiofrequency (RF) telemetry and inductive coupling, but other techniques are also contemplated. In some examples, programmer 150 may include a communication head that may be placed proximate to the patient's body near the IMD 110 implant site to improve the quality or security of communication between IMD 110 and programmer 150. Communication between programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted) on leads 130. In some examples, IMD 110 may modify therapy stimulation programs as therapy needs of patient 105 evolve over time. For example, the modification of the therapy stimulation programs may cause the adjustment of at least one parameter of the plurality of informed pulses. When patient 105 receives the same therapy for an extended period, the efficacy of the therapy may be reduced. In some cases, parameters of the plurality of informed pulses may be automatically updated.

In this disclosure, efficacy of SCS therapy may be indicated by one or more characteristics of an action potential that is evoked by a stimulation pulse delivered by IMD 110 (i.e., a characteristic of the ECAP signal). Various non-limiting examples of identifiable characteristics of ECAP signals include a signal amplitude (e.g., an amplitude of or between one or more signal peaks such as any combination of the P1, N1, P2, N2, P3, or other peaks), a signal width, a signal latency (e.g., a duration between delivery of the control pulse and sensing of the ECAP signal), a signal slope, an area-under-the-curve (AUC) of one or more peaks, a signal phasing, a signal curvature, an oscillatory signal pattern, a signal morphology; or a temporal stability or temporal variance of the ECAP signal.

In some examples, IMD 110 is configured to measure or sense the ECAP signal on a substantially continuous basis, e.g., in examples in which the stimulation therapy includes a generally continuous waveform rather than a discrete pulse train. In other examples, IMD 110 is configured to measure or sense the ECAP signal in response to the delivery of each informed stimulation pulse. In other examples, IMD 110 is configured to “sample” the ECAP signal at a predetermined sampling frequency, such as about 50 Hz, as one non-limiting example.

Electrical-stimulation-therapy delivery by leads 130 of IMD 110 may cause neurons within the target tissue to evoke a compound action potential that travels up and down the target tissue, eventually arriving at sensing electrodes of IMD 110. Furthermore, control stimulation may also elicit at least one ECAP, and ECAPs responsive to control stimulation may also be a surrogate for the effectiveness of the therapy. The number of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. The slew rate may define the rate of change of the voltage and/or current amplitude of the pulse at the beginning and/or end of each pulse or each phase within the pulse. For example, a very high slew rate indicates a steep or even near vertical edge of the pulse, and a low slew rate indicates a longer ramp up (or ramp down) in the amplitude of the pulse. In some examples, these parameters may contribute to an intensity of the electrical stimulation. In addition, a characteristic of the ECAP signal (e.g., an amplitude) may change based on the distance between the stimulation electrodes and the nerves subject to the electrical field produced by the delivered control pulses.

In one example, each informed pulse may have a pulse width greater than approximately 100 μs, such as between approximately 200 μs and 1000 μs (i.e., 1 millisecond) in some examples. At these pulse widths, IMD 110 may not sufficiently detect an ECAP signal because the informed pulse is also detected as an artifact that obscures the ECAP signal. If ECAPs are not adequately recorded, then ECAPs arriving at IMD 110 cannot be compared to the target ECAP characteristic (e.g. a target ECAP amplitude), and electrical therapy stimulation cannot be altered according to responsive ECAPs. When informed pulses have these longer pulse widths, IMD 110 may deliver control stimulation in the form of control pulses. The control pulses may have pulse widths of less than approximately 300 μs, such as a bi-phasic pulse with each phase having a duration of approximately 170 μs in one example. Since the control pulses may have shorter pulse widths than the informed pulses, the ECAP signal may be sensed and identified following each control pulse and used to inform IMD 110 about any changes that should be made to the informed pulses (and control pulses in some examples). In some examples, at least some informed pulses may have pulse widths less than approximately 300 μs, such as 200 μs. In such examples, control pulses interleaved with the informed pulses may have pulse widths shorter than the pulse widths of informed pulses. In other examples, a control pulse may have a pulse width greater than the pulse width of the informed pulse. In general, the term “pulse width” refers to the collective duration of every phase, and interphase interval when appropriate, of a single pulse. A single pulse may include a single phase in some examples (i.e., a monophasic pulse) or two or more phases in other examples (e.g., a bi-phasic pulse or a tri-phasic pulse). The pulse width defines a period of time beginning with a start time of a first phase of the pulse and concluding with an end time of a last phase of the pulse (e.g., a biphasic pulse having a positive phase lasting 100 μs, a negative phase lasting 100 μs, and an interphase interval lasting 30 μs defines a pulse width of 230 μs). In some examples, the informed pulses may consist of a train of multiple consecutive pulses with a parameter set (pulse width, amplitude, pulse-to-pulse period, morphology) informed by a preceding control pulse. In other words, in some cases, a plurality of consecutive informed pulses may be interleaved between a pair of discrete control pulses. In this manner, one or more informed pulses may be delivered between each control pulse of a train of control pulses. The system may adjust this ratio of informed pulses to control pulses as needed, e.g., in response to one or more factors associated with therapy, ECAP sensing, and/or system characteristics such as battery life (e.g., fewer delivered pulses increases battery life).

As described, the example techniques for adjusting stimulation parameter values for informed pulses are based on comparing the value of a characteristic of a measured ECAP signal to a target ECAP characteristic value. During delivery of control pulses defined by one or more ECAP test stimulation programs, IMD 110, via two or more electrodes interposed on leads 130, senses electrical potentials of tissue of the spinal cord 120 of patient 105 to measure the electrical activity of the tissue. IMD 110 senses ECAPs from the target tissue of patient 105, e.g., with electrodes on one or more leads 130 and associated sense circuitry. In some examples, IMD 110 receives a signal indicative of the ECAP from one or more sensors, e.g., one or more electrodes and circuitry, internal or external to patient 105. Such an example signal may include a signal indicating an ECAP of the tissue of the patient 105. Examples of the one or more sensors include one or more sensors configured to measure a compound action potential of the patient 105, or a physiological effect indicative of a compound action potential. For example, to measure a physiological effect of a compound action potential, the one or more sensors may be an accelerometer, a pressure sensor, a bending sensor, a sensor configured to detect a posture of patient 105, or a sensor configured to detect a respiratory function of patient 105. However, in other examples, external programmer 150 receives a signal indicating a compound action potential in the target tissue of patient 105 and transmits a notification to IMD 110.

In the example of FIG. 1, IMD 110 described as performing a plurality of processing and computing functions. However, external programmer 150 instead may perform one, several, or all of these functions. In this alternative example, IMD 110 functions to relay sensed signals to external programmer 150 for analysis, and external programmer 150 transmits instructions to IMD 110 to adjust the one or more parameters defining the electrical stimulation therapy based on analysis of the sensed signals. For example, IMD 110 may relay the sensed signal indicative of an ECAP to external programmer 150. External programmer 150 may compare the parameter value of the ECAP to the target ECAP characteristic value, and in response to the comparison, external programmer 150 may instruct IMD 110 to adjust one or more parameter that defines the electrical stimulation informed pulses and, in some examples, control pulses, delivered to patient 105.

In the example techniques described in this disclosure, the control stimulation parameters and the target ECAP characteristic values may be initially set at the clinic but may be set and/or adjusted at home by patient 105. Once the target ECAP characteristic values are set, the example techniques allow for automatic adjustment of informed pulse parameters to maintain consistent volume of neural activation and consistent perception of therapy for the patient when the electrode-to-neuron distance changes. The ability to change the stimulation parameter values may also allow the therapy to have long term efficacy, with the ability to keep the intensity of the stimulation (e.g., as indicated by the ECAP) consistent by comparing the measured ECAP values to the target ECAP characteristic value. IMD 110 may perform these changes without intervention by a physician or patient 105.

In some examples, SCS system 100 is configured to periodically store sensed ECAP signal parameters in memory. SCS system 100 may use the stored ECAP parameters to monitor a neurophysiologic state of the patient over time, e.g., to enable long-term monitoring of the progression of the disease, patient condition, and/or efficacy of therapy.

As described herein, system 100 can be configured to determine a stimulus schedule (e.g., schedule of stimulation pulses) based on identified artifacts in sensed electrical signals. For example, processing circuitry of system 100 (e.g., processing circuitry of IMD 110 and/or programmer 150) can control stimulation generation circuitry (e.g., stimulation circuitry 214 of FIG. 2A) to deliver at least one first pulse from a first electrode combination and control sensing circuitry (e.g., sensing circuitry 212) to sense an electrical signal during a sensing window associated with delivery of a second pulse from a second electrode combination different than the first electrode combination. In this manner, the sensing window may be initially set to a predetermined, or preliminary, time period after the second pulse is delivered from the second electrode combination. In some examples, this second pulse may be referred to as a “control pulse” if the system is configured to detect an evoked signal elicited by the first pulse.

Processing circuitry of system 100 can then generate an analysis of the electrical signal sensed during the sensing window. The analysis performed by the processing circuitry may be configured to identify one or more artifacts associated with the at least one first stimulus from the first electrode combination. These artifacts may include one or more signals representative of the first pulse itself and/or evoked signals that may have been elicited by the first pulse. The processing circuitry can then determine, based on the analysis, a stimulus schedule for a plurality of subsequent pulses deliverable from the first electrode combination. This stimulus schedule may thus be a modified version of one or more trains of pulses initially programmed to be delivered by the first electrode combination. As described herein, the processing circuitry may generate the stimulus schedule by removing, moving, adding, or otherwise adjusting one or more pulse trains that may be programmed to be delivered from the first electrode combination. In some examples, the stimulus schedule may define one or more stimulation pulses from more than one electrode combination in order to reduce or eliminate artifacts from the sensing window.

FIG. 2A is a block diagram illustrating some example components of an IMD 200, which is an example of IMD 110 of FIG. 1. In the example shown in FIG. 2A, IMD 200 includes processing circuitry 214, memory 215, stimulation generator 211, sensing circuitry 212, telemetry module 213, sensor 216, and power source 219. Each of these modules may be or may include programmable or fixed-function circuitry configured to perform the functions attributed to respective circuitry. For example, processing circuitry 214 may include fixed-function or programmable circuitry; stimulation generator 211 may include circuitry configured to generate stimulation signals such as pulses or continuous waveforms on one or more channels; sensing circuitry 212 may include sensing circuitry for sensing signals, and telemetry module 213 may include telemetry circuitry for transmission and reception of signals. Memory 215 may store computer-readable instructions that, when executed by processing circuitry 214, cause IMD 200 to perform various functions described herein. Memory 215 may be a storage device or other non-transitory medium.

In the example shown in FIG. 2A, memory 215 stores therapy stimulation programs 217 and ECAP test stimulation programs 218 in separate (e.g., physically distinct) memories within memory 215 or separate areas (e.g., partitions) within memory 215. Memory 215 also stores target ECAP feedback rules 221 and patient ECAP characteristics 222. Each stored therapy stimulation programs 217 defines values for a set of electrical stimulation parameters (e.g., a parameter set or set of parameter values), such as a stimulation electrode combination, electrode polarity, current or voltage amplitude, pulse width, pulse rate, and pulse shape. Each of stored ECAP test stimulation programs 218 defines values for a corresponding set of electrical stimulation parameters (e.g., a control stimulation parameter set), such as a stimulation electrode combination, electrode polarity, current or voltage amplitude, pulse width, pulse rate, and pulse shape. ECAP test stimulation programs 218 may also have additional information such as instructions regarding when to deliver control pulses based on the pulse width and/or frequency of the informed pulses defined in therapy stimulation programs 217. Determining a stimulus schedule for stimulation may include modifying one or more programs of therapy stimulation programs 217 and/or ECAP test stimulation programs 218.

Accordingly, in some examples, stimulation generator 211 generates electrical stimulation signals in accordance with the electrical stimulation parameters noted above. Other ranges of parameter values may also be useful and may depend on the target stimulation site within patient 105. While stimulation pulses are described, stimulation signals may be of any form, such as time-continuous signals (e.g., sinusoidal waves) or the like.

In some examples, IMD 200 may include independently controllable current sources and sinks coupled to individual electrodes 232, 234. For instance, stimulation generator 211 may include an array of regulated current sources and sinks, coupled (via implantable leads 230) to respective individual electrodes 232, 234, that can be selectively activated to form electrode combinations and deliver stimulation pulses. In some such examples, to interleave control stimulation pulses with informed stimulation pulses, IMD 200 may further include one or more gate transistors to actuate the current sources or sinks and/or to toggle between stimulation generator 211 and sensing circuitry 212.

For example, to activate electrode 232A as a cathode, processing circuitry 214 may turn on a current source (e.g., of stimulation generator 211) that is connected to electrode 232A and specify an amount of electric current amplitude to be delivered with each pulse. To activate electrode 232B as an anode, processing circuitry 214 may turn on a current sink (e.g., of stimulation generator 211) connected to electrode 232B, which causes electrode 232B to sink the amount of current sourced by electrode 232A. IMD 200 can time-mux different electrode combinations by turning on different sources and sinks of stimulation generator 211, connected to other selected electrodes, at different times. IMD 200 can also form multi-electrode combinations of multiple cathodes and/or multiple anodes (or one cathode and multiple anodes or one anode and multiple cathodes) by selectively turning on particular sources and sinks, with the total amount of current sourced by the current sources of the cathodes being equal to the total amount of current sunk by the current sinks of the anode.

In some examples, each current source or sink of stimulation generator 211 may be or may include a set of multiple, parallel sources and sinks formed by branches of a current mirror circuit. IMD 200 may selectively activate the parallel sources and sinks to dial up or down the amount of current for a given electrode 232, 234. For example, if there are 64 parallel source branches, activating 32 of them provides 32 times the current delivered in the reference branch of the current mirror circuit, e.g., 50% of the maximum possible current that could be delivered (e.g., if all 64 branches were activated) by the electrode 232, 234. In some instances, the reference current may also be adjustable. In some examples, the outer housing of IMD 200 may also form an anode for some applications.

Processing circuitry 214 may include any one or more of a microprocessor, a controller, a digital-signal processor (DSP), an application-specific integrated circuit (ASIC), a field-programmable gate array (FPGA), discrete logic circuitry, or any other processing circuitry configured to provide the functions attributed to processing circuitry 214 herein, and may be embodied as firmware, hardware, software, or any combination thereof. Processing circuitry 214 controls stimulation generator 211 to generate stimulation signals according to therapy stimulation programs 217 and ECAP test stimulation programs 218 stored in memory 215 to apply stimulation parameter values specified by one or more of programs, such as amplitude, pulse width, pulse rate, and pulse shape of each of the stimulation signals.

In the example shown in FIG. 2A, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 214 also controls stimulation generator 211 to generate and apply the stimulation signals to selected combinations of electrodes 232, 234. In some examples, stimulation generator 211 includes a switch circuit that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord 120 of the patient 105 (FIG. 1) with selected electrodes 232, 234.

In other examples, however, stimulation generator 211 does not include a switch circuit and, as described above, stimulation generator 211 comprises a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generator 211 via respective wires that are straight or coiled within the housing of the lead 230 and run to a connector at the proximal end of the lead. In another example, each of the electrodes 232, 234 of the lead 230 may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 212 is incorporated into a common housing with stimulation generator 211 and processing circuitry 214 in FIG. 2A, in other examples, sensing circuitry 212 may be in a separate housing from IMD 200 and may communicate with processing circuitry 214 via wired or wireless communication techniques.

In some examples, one or more of electrodes 232 and 234 may be suitable for sensing the ECAPs. For instance, electrodes 232 and 234 may sense the voltage amplitude of a portion of the ECAP signals, where the sensed voltage amplitude is a characteristic the ECAP signal.

Sensor 216 may include one or more sensing elements that sense values of a respective patient parameter. As described, electrodes 232 and 234 may be the electrodes that sense the parameter value of the ECAP. Sensor 216 may include one or more accelerometers, optical sensors, chemical sensors, temperature sensors, pressure sensors, EEG sensors, EMG sensors, or any other types of sensors. Sensor 216 may output patient parameter values that may be used as feedback to control delivery of SCS therapy.

Telemetry module 213 supports wireless communication between IMD 200 and an external programmer 300 (FIG. 2B) or another computing device under the control of processing circuitry 214. Processing circuitry 214 of IMD 200 may receive, as updates to programs, values for various stimulation parameters such as amplitude and electrode combination, from the external programmer via telemetry module 213. Updates to the therapy stimulation programs 217 and ECAP test stimulation programs 218 may be stored within memory 215. Telemetry module 213 in IMD 200, as well as telemetry circuits in other devices and systems described herein, such as the external programmer, may accomplish communication by radiofrequency (RF) communication techniques. In addition, telemetry module 213 may communicate with an external medical device programmer (not shown in FIG. 2A) via proximal inductive interaction of IMD 200 with the external programmer. The external programmer may be one example of external programmer 150 of FIG. 1. Accordingly, telemetry module 213 may send information to the external programmer on a continuous basis, at periodic intervals, or upon request from IMD 110 or the external programmer.

Power source 219 delivers operating power to various components of IMD 200. Power source 219 may include a rechargeable or non-rechargeable battery and a power-generation circuit to produce the operating power. Recharging may be accomplished through proximal inductive interaction between an external charger and an inductive charging coil within IMD 200. In other examples, traditional primary cell batteries may be used.

According to the techniques of the disclosure, stimulation generator 211 of IMD 200 receives, via telemetry module 213, instructions to deliver SCS therapy according to therapy stimulation programs 217 to at least one target tissue site located on the spinal cord via a plurality of electrode combinations of electrodes 232, 234 of leads 230 and/or a housing of IMD 200. Stimulation generator 211 may receive, via telemetry module 213, user instructions to deliver control stimulation to the patient according to ECAP test stimulation programs 218. Each pulse of a plurality of control pulses may elicit an ECAP that is sensed by sensing circuitry 212 via some of electrodes 232 and 234. ECAP test stimulation programs 218 may instruct stimulation generator 211 to deliver a plurality of control pulses interleaved with at least some of the plurality of informed pulses. Processing circuitry 214 may receive, via an electrical signal sensed by sensing circuitry 212, information indicative of an ECAP signal (e.g., a numerical value indicating a characteristic of the ECAP in electrical units such as voltage or power) produced in response to the control stimulation. Therapy stimulation programs 217 may be updated according to the ECAPs recorded at sensing circuitry 212 according to the following techniques.

In one example, the plurality of informed pulses each have a pulse width of greater than approximately 100 μs and less than approximately 2000 μs (i.e., 2 milliseconds). In some examples, the informed pulse width is greater than approximately 200 μs and less than approximately 800 μs. In another example, the informed pulse width is greater than approximately 300 μs and less than approximately 500 μs. In one example, informed pulses have a pulse width of approximately 450 μs and a pulse frequency of approximately 60 Hertz. Amplitude (current and/or voltage) for the informed pulses may be between approximately 0.5 mA (or volts) and approximately 10 mA (or volts), although amplitude may be lower or greater in other examples. In some examples, the pulse width of the informed pulses may be less than 300 μs. In some examples, the system may deliver informed pulses from two or more stimulation programs such that the informed pulses from one stimulation program have at least one different parameter value than the informed pulses from another stimulation program. As described above, the plurality of informed pulses may include multiple consecutive, discrete informed pulses delivered between a pair of discrete control pulses. In this manner, the system may be configured to deliver a train of control pulses, where one, two, three, four, or more informed pulses are delivered between consecutive control pulses of the train of control pulses. Each of the informed pulses may define a pulse width that is shorter or longer than a pulse width of the control pulses.

Each control pulse of the plurality of control pulses may have a pulse width of less than approximately 300 μs. In one example, each control pulse of the plurality of control pulses may be a bi-phasic pulse with a positive phase having a width of approximately 100 μs, a negative phase having a width of approximately 100 μs, and an interphase interval having a width of approximately 30 μs. In some examples, the positive phase and negative phase may each be 90 μs or 120 μs in other examples. In other examples, the control pulses may each have a pulse width of approximately 170 μs, 60 μs, or smaller. Due to the relatively long pulse widths of the plurality of informed pulses and/or higher frequency of informed pulse delivery, sensing circuitry 212 may be incapable of adequately recording an ECAP signals elicited from an informed pulse because the informed pulse itself will occur during the ECAP signal and obscure the ECAP signal. However, stimulation pulses with pulse widths less than approximately 300 microseconds, such as the plurality of control pulses, may be suited to elicit an ECAP which can be sensed after the control pulse is completed at sensing circuitry 212 via two or more of electrodes 232 and 234. In some examples, the control pulses may be non-therapeutic pulses in that the control pulses do not contribute to therapy for the patient. In other examples, the control pulses may fully provide or partially contribute to the therapy received by the patient by reducing or eliminating symptoms and/or a condition of the patient.

Control pulses delivered for the purpose of eliciting detectable ECAP signals may have a current amplitude between approximately 1 mA and 12 mA in some examples, but higher or lower amplitudes may be used in other examples. The frequency of the control pulses may be between approximately 40 Hertz and 400 Hertz in some examples, which may match the predetermined pulse frequency of the informed pulses when one control pulse is delivered for each therapeutic pulse. The predetermined pulse frequency may be a single frequency or a varied frequency over time (e.g., the interpulse interval may change over time according to predetermined pattern, formula, or schedule). In some examples, the system may change the predetermined pulse frequency based on patient input or a sensed parameter such as patient posture or activity. Such a relationship may be present when the control pulses are fully interleaved (e.g., alternating) with the informed pulses. However, the frequency of the control pulses may be delivered at a higher frequency than then informed pulses when two or more control pulses are delivered between consecutive informed pulses. In other examples, the frequency of the control pulses may be delivered at a lower frequency than the informed pulses when at least some informed pulses are delivered without a control pulse delivered between them. The frequency of the control pulses may be delivered at a frequency that varies over time if the system is configured to adjust control pulse delivery, and the resulting ECAP sensing, based on other factors such as patient activity.

In one example, the predetermined pulse frequency of the plurality of informed pulses may be less than approximately 400 Hertz. In some examples, the predetermined pulse frequency of the plurality of informed pulses may be between approximately 40 Hertz and 70 Hertz. In one example, the predetermined pulse frequency of the plurality of informed pulses may be approximately 50 Hertz. However, the informed pulses may have frequencies greater than 400 Hertz or less than 40 Hertz in other examples. In some examples, the predetermined pulse frequency of the informed pulses may be a single frequency or a frequency that varies over time. In addition, the informed pulses may be delivered in bursts of pulses, with interburst frequencies of the pulses being low enough such that a control pulse and sensed ECAP can still fit within the window between consecutive informed pulses delivered within the burst of informed pulses.

Since each informed pulse of the plurality of informed pulses may be sensed as an artifact that covers, or obscures, the sensing of at least one ECAP, the plurality of control pulses may be delivered to the patient during a plurality of time events. For example, a time event (e.g., a sense window) of the plurality of time events may be a time (e.g., a sense window) between consecutive informed pulses of the plurality of informed pulses at the predetermined pulse frequency. One or more control pulses of the plurality of control pulses may be delivered to the patient during each time event. Consequently, the control pulses may be interleaved with at least some of the informed pulses such that the plurality of control pulses are delivered to the patient while informed pulses are not delivered. In one example, an ECAP elicited from to a control pulse delivered during a time event may be recorded by sensing circuitry 212 during the same sensing window. In another example, two or more ECAPs responsive to two or more respective control pulses delivered during a time event may be recorded by sensing circuitry 212 during the same time event. However, as described herein, the system may not be able to accurately predict when stimulation pulses do not interfere with the sensing window. Therefore, the system may attempt to identify any artifacts within the sensing window and adjust the stimulus schedule as needed (e.g., remove, more, or even add) stimulation pulses in order to reduce the likelihood of artifacts from occurring within the sensing window.

In some examples, therapy stimulation programs 217 may be updated according to a plurality of ECAPs received in response to the plurality of control pulses delivered to the patient according to ECAP test stimulation programs 218. For instance, processing circuitry 214 may update therapy stimulation programs 217 in real time by comparing one or more characteristics of ECAPs sensed by sensing circuitry 212 with target ECAP characteristics stored in memory 215 (e.g., patient ECAP characteristics 222). For example, processing circuitry 214 is configured to determine the amplitude of each ECAP signal received at sensing circuitry 212, and processing circuitry 214 is further configured to determine the representative amplitude of at least one respective ECAP signal and compare the representative amplitude of a series of ECAP signals to a target ECAP adjustment window (e.g., the target ECAP amplitude plus and minus a variance which is stored in patient ECAP characteristics 222). The target ECAP adjustment window may thus be a range of amplitudes deviating from target ECAP amplitude. For instance, the target ECAP adjustment window may span from a lower-bound amplitude value (e.g., the target ECAP amplitude minus the variance) to an upper-bound amplitude value (e.g., the target ECAP amplitude plus the variance). Generally, the lower-bound amplitude value is less than the target ECAP amplitude, and the upper-bound amplitude value is greater than target ECAP amplitude.

If the representative amplitude of the at least one respective ECAP signal (e.g., an amplitude of a single ECAP signal or an average of two or more ECAP amplitudes) is greater than the upper-bound amplitude value, processing circuitry 214 may adjust one or more of therapy stimulation programs 217 and ECAP test stimulation programs 218 to decrease the amplitude of informed pulses and control pulses following the at least one respective ECAP. The amplitude of informed pulses and control pulses may be decreased by different predetermined steps or different predetermined percentages. Additionally, if the representative amplitude of the at least one respective ECAP is less than the lower-bound amplitude value, processing circuitry 214 may adjust therapy stimulation programs 217 and ECAP test stimulation programs 218, and the programs 217 and 218 may instruct stimulation generator 211 to increase the amplitude of informed pulses and control pulses following the at least one respective ECAP. Moreover, if the representative amplitude of the at least one respective ECAP is greater than the lower-bound amplitude value and less than the upper-bound amplitude value, processing circuitry 214 may not change programs 217 and 218, and stimulation generator 211 may maintain the amplitude of the informed pulses following the at least one respective ECAP. In one example, adjusting the programs 217 and 218 may include changing one or more parameters of the plurality of informed pulses and the plurality of control pulses. In one example, the at least one respective ECAP may include a series of four consecutive ECAPs.

Processing circuitry 214, in one example, may change the amplitude of the informed pulses and the control pulses following the at least one respective ECAP inversely proportional to the difference between target ECAP amplitude and the representative amplitude of the at least one respective ECAP. For instance, if the representative amplitude of the at least one respective ECAP is 20% lower than the target ECAP amplitude, then processing circuitry 214 may update therapy programs 217 and 218 such that the amplitude of informed pulses and the control pulses is increased by 20%. In one example, the representative amplitude may be the mean amplitude of two or more respective ECAP signals sensed by sensing circuitry 212. In other examples, the representative amplitude may be the median amplitude of two or more respective ECAP signals, or a rolling average of two or more respective ECAP signals.

In another example, processing circuitry 214 may determine the amplitude of a respective ECAP signal sensed by sensing circuitry 212. In response to a comparison between the amplitude of the respective ECAP signal and the target ECAP amplitude stored in patient ECAP characteristics 222, processing circuitry 214 may determine a percentage difference between the amplitude of the respective ECAP signal and target ECAP amplitude. Consequently, processing circuitry 214 may adjust the amplitude of subsequent informed pulses to be inversely proportional to the percentage difference between the amplitude of the respective ECAP and target ECAP amplitude.

In other examples, processing circuitry 214 may use the representative amplitude of the at least one respective ECAP to change other parameters of informed pulses to be delivered, such as pulse width, pulse frequency, and pulse shape. All of these parameters may contribute to the intensity of the informed pulses, and changing one or more of these parameter values may effectively adjust the informed pulse intensity to compensate for the changed distance between the stimulation electrodes and the nerves indicated by the representative amplitude of the ECAP signals.

In some examples in which leads 230 include linear 8-electrode leads (not pictured in FIG. 2A), sensing and stimulation delivery may each be performed using a different set of electrodes. For instance, in a linear 8-electrode lead, each electrode may be numbered consecutively from 0 through 7. A control pulse may be generated using electrode 1 as a cathode and electrodes 0 and 2 as anodes (e.g., a guarded cathode), and a respective ECAP signal may be sensed using electrodes 6 and 7, located on the opposite end of the electrode array. This strategy may minimize the interference of the stimulation pulse with the sensing of the respective ECAP. Other electrode combinations may be implemented, and the electrode combinations may be changed using the patient programmer via telemetry module 213. For example, stimulation electrodes and sensing electrodes may be positioned closer together. Shorter pulse widths for the control pulses may allow the sensing electrodes to be closer to the stimulation electrodes.

ECAP feedback rules 221 may define how processing circuitry 214 uses the sensed ECAP signals as feedback for changing one or more parameters that define informed pulses and stored as therapy stimulation programs 217. For example, ECAP feedback rules 221 may specify that the percentage difference between the representative ECAP amplitude and the target ECAP amplitude is used to inversely adjust the current amplitude of informed pulses to the same proportion as the percentage difference. As another example, ECAP feedback rules 221 may specify that the difference between the target ECAP amplitude is multiplied by a gain value and added to the previous current amplitude of the informed pulses and control pulses. In any case, ECAP feedback rules 221 may instruct processing circuitry 14 how to adjust informed pulses and/or control pulses based on the sensed ECAP signals. In other examples, ECAP feedback rules 221 may instruct processing circuitry 214 to adjust an amplitude of stimulation pulses, or start or stop deliver of stimulation, in response to detecting the presence or absence of ECAP signals (e.g., an ECAP characteristic value above or below a minimum threshold or perception threshold).

FIG. 2B is a block diagram illustrating some example components of an external programmer 300, which is an example of external programmer 150 of FIG. 1. Although programmer 300 may generally be described as a “handheld” device, programmer 300 may be a larger portable device or a more stationary device. In addition, in other examples, programmer 300 may be included as part of an external charging device or include the functionality of an external charging device. As illustrated in FIG. 2B, programmer 300 may include processing circuitry 353, a memory 354, a user interface 351, telemetry module 352, and a power source 355. Memory 354 may store instructions that, when executed by processing circuitry 353, cause processing circuitry 353 and external programmer 300 to provide the functionality ascribed to external programmers 150, 300 throughout this disclosure. Each of these components, circuitry, or modules, may include electrical circuitry that is configured to perform some, or all of the functionality described herein. For example, processing circuitry 353 may include processing circuitry configured to perform the processes discussed with respect to processing circuitry 353.

In general, programmer 300 comprises any suitable arrangement of hardware, alone or in combination with software and/or firmware, to perform the techniques attributed to programmer 300, and processing circuitry 353, user interface 351, and telemetry circuitry 352 of programmer 300. In various examples, programmer 300 may include one or more processors, such as one or more microprocessors, DSPs, ASICs, FPGAs, or any other equivalent integrated or discrete logic circuitry, as well as any combinations of such components. Programmer 300 also, in various examples, may include a memory 354, such as RAM, ROM, PROM, EPROM, EEPROM, flash memory, a hard disk, a CD-ROM, comprising executable instructions for causing the one or more processors to perform the actions attributed to them. Moreover, although processing circuitry 353 and telemetry circuitry 352 are described as separate modules, in some examples, processing circuitry 353 and telemetry circuitry 352 are functionally integrated. In some examples, processing circuitry 353 and telemetry circuitry 352 correspond to individual hardware units, such as ASICs, DSPs, FPGAs, or other hardware units.

Memory 354 (e.g., a storage device) may store instructions that, when executed by processing circuitry 353, cause processing circuitry 353 and programmer 300 to provide the functionality ascribed to programmers 150, 300 throughout this disclosure. For example, memory 354 may include instructions that cause processing circuitry 353 to obtain a parameter set from memory, select a spatial electrode movement pattern, or receive a user input and send a corresponding command to IMD 300, or instructions for any other functionality. In addition, memory 354 may include a plurality of programs, where each program includes a parameter set that defines therapy stimulation or control stimulation. Memory 354 may also store data received from a medical device (e.g., IMD 110). For example, memory 354 may store ECAP related data recorded at a sensing module of the medical device, and memory 354 may also store data from one or more sensors of the medical device.

User interface 351 may include a button or keypad, lights, a speaker for voice commands, a display, such as a liquid crystal (LCD), light-emitting diode (LED), or organic light-emitting diode (OLED). In some examples the display may be a touch screen. User interface 351 may be configured to display any information related to the delivery of electrical stimulation, identified patient behaviors, sensed patient parameter values, patient behavior criteria, or any other such information. User interface 351 may also receive user input via user interface 351. The input may be submitted by a user, for example, by pressing a button on a keypad or by selecting an icon from a touch screen. The input may request, as non-limiting examples, the starting or stopping of SCS therapy delivery, a new spatial electrode movement pattern or a change to an existing spatial electrode movement pattern, or some other change to the delivery of electrical stimulation.

Telemetry module 352 may support wireless communication between the medical device 110 of FIG. 1 and programmer 300 under the control of processing circuitry 353. Telemetry module 352 may also be configured to communicate with another computing device via wireless communication techniques, or direct communication through a wired connection. In some examples, telemetry module 352 provides wireless communication via an RF or proximal inductive medium. In some examples, telemetry module 352 includes an antenna, which may take on a variety of forms, such as an internal and/or external antenna.

Examples of local wireless communication techniques that may be employed to facilitate communication between programmer 300 and IMD 110 of FIG. 1 include RF communication according to the 802.11 or Bluetooth specification sets or other standard or proprietary telemetry protocols. In this manner, other external devices may be capable of communicating with programmer 300 without needing to establish a secure wireless connection. As described herein, telemetry circuitry 352 may be configured to transmit a spatial electrode movement pattern or other stimulation parameter values to IMD 110 for delivery of electrical stimulation therapy.

In some examples, selection of parameters or therapy stimulation programs may be transmitted to medical device 110 for delivery to patient 105. In other examples, the therapy may include medication, activities, or other instructions that patient 105 must perform themselves or that a caregiver must perform for patient 105. In some examples, programmer 300 may provide visual, audible, and/or tactile notifications indicating that there are new instructions. Programmer 300 may require the receipt of user input acknowledging that the instructions have been completed, in some examples.

According to techniques of the disclosure, user interface 351 of external programmer 300 receives an indication, e.g., from a clinician, instructing a processor 214 of medical device 200 (FIG. 2A) to update one or more therapy stimulation programs 217 or to update one or more ECAP test stimulation programs 218. Updating therapy stimulation programs 217 and ECAP test stimulation programs 218 may include changing one or more parameters of the stimulation pulses delivered by medical device 200 according to the programs, such as amplitude, pulse width, frequency, pulse shape, or other parameters of the informed pulses and/or control pulses. User interface 351 may also receive instructions from the clinician commanding any electrical stimulation, including therapy stimulation and control stimulation to commence or to cease.

The architecture of programmer 300 illustrated in FIG. 2B is shown as an example. The techniques as set forth in this disclosure may be implemented in the example programmer 300 of FIG. 2B, as well as in other types of systems not described specifically herein. Nothing in this disclosure should be construed so as to limit the techniques of this disclosure to the example architecture illustrated by FIG. 2B.

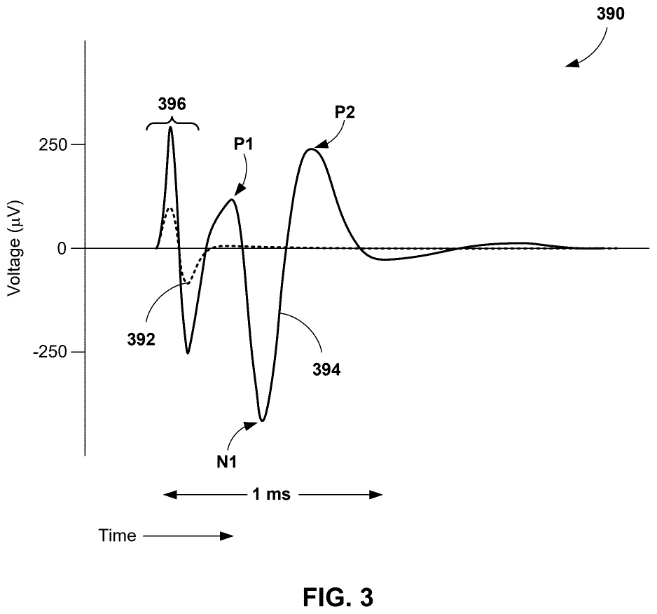

FIG. 3 is a graph 390 of an example of evoked compound action potential (ECAP) sensed for respective stimulation pulses. As shown in FIG. 3, graph 390 shows example signal 392 (dotted line) and ECAP signal 394 (solid line). Each of signals 392 and 394 may be sensed from control pulses that were delivered from a guarded cathode and bi-phasic pulses including an interphase interval between each positive and negative phase of the pulse. The guarded cathode of the stimulation electrodes may be located at one end of an 8-electrode lead, while two sensing electrodes are provided at the opposite end of the 8-electrode lead. Signal 392 illustrates the sensed voltage amplitude resulting from a sub-threshold stimulation pulse. In other words, although peaks 396 of signal 392 are detected and represent the artifact of the delivered control pulse. However, no propagating signal is detected after the artifact in signal 392 because the control pulse was sub-threshold. Whether or not an ECAP signal is generated from a stimulus, the stimulus may still be detectable by sensing electrodes depending on the timing of the stimulus and distance to the sensing electrodes.

In contrast to signal 392, ECAP signal 394 represents the sensed voltage amplitude resulting from a supra-threshold control pulse. Peak(s) 396 of ECAP signal 394 are detected and represent the artifact of the delivered control pulse. After peak(s) 396, ECAP signal 394 also includes peaks P1, N1, and P2, which are three typical peaks representative of propagating action potentials from an ECAP. The example duration of the artifact and peaks P1, N1, and P2 is approximately 1 millisecond (ms). When detecting the ECAP of ECAP signal 394, different characteristics may be identified. For example, the characteristic of the ECAP may be the amplitude between N1 and P2. This N1-P2 amplitude may be easily detectable even if the artifact impinges on P1, a relatively large signal, and the N1-P2 amplitude may be minimally affected by electronic drift in the signal. In other examples, the characteristic of the ECAP used to control informed pulses may be an amplitude of P1, N1, or P2 with respect to a neutral (e.g., zero) voltage. In some examples, the characteristic of the ECAP used to control informed pulses may be a sum of two or more of peaks P1, N1, or P2. In other examples, the characteristic of ECAP signal 394 may be the area under one or more of peaks P1, N1, and/or P2. In other examples, the characteristic of the ECAP may be a ratio of one of peaks P1, N1, or P2 to another one of the peaks. In some examples, the characteristic of the ECAP may be a slope between two points in the ECAP signal, such as the slope between N1 and P2. In other examples, the characteristic of the ECAP may be the time between two points of the ECAP, such as the time between N1 and P2. The time between two points in the sensed ECAP signal may be referred to as a “latency” of the ECAP and may indicate the types of fibers being captured by the control pulse. ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Other characteristics of the ECAP signal may be used in other examples.

The amplitude of the ECAP signal increases with increased amplitude of the control pulse, as long as the pulse amplitude is greater than threshold such that nerves depolarize and propagate the signal. The target ECAP characteristic (e.g., the target ECAP amplitude) may be determined from the ECAP signal detected from a control pulse when informed pulses are determined to deliver effective therapy to the patient. The ECAP signal thus is representative of the distance between the stimulation electrodes and the nerves appropriate for the stimulation parameter values of the informed pulses delivered at that time. Therefore, IMD 110 may attempt to use detected changes to the measured ECAP characteristic value to change informed pulse parameter values and maintain the target ECAP characteristic value during informed pulse delivery.

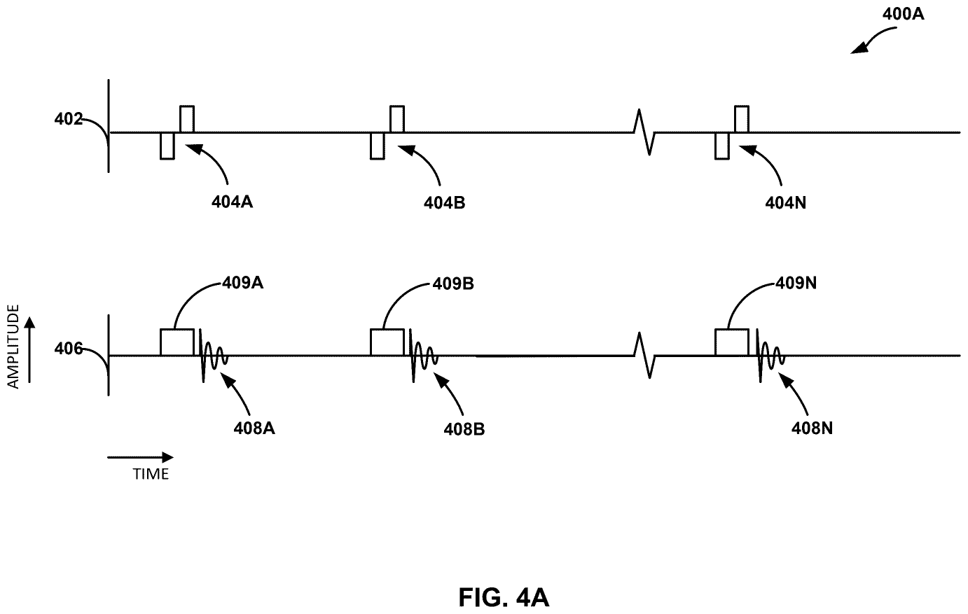

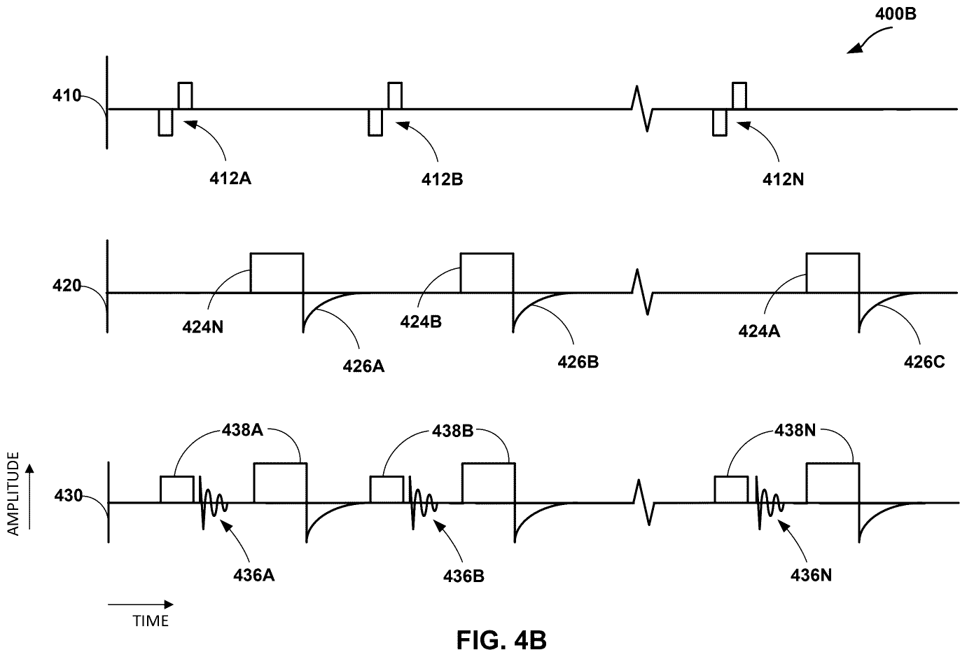

FIG. 4A is a timing diagram 400A illustrating an example of electrical stimulation pulses and respective sensed ECAP signals, in accordance with one or more techniques of this disclosure. For example, FIG. 4A is described with reference to IMD 200 of FIG. 2A. As illustrated, timing diagram 400A includes first channel 402, a plurality of control pulses 404A-404N (collectively “control pulses 404”), second channel 406, a plurality of respective ECAPs 408A-408N (collectively “ECAPs 408”), and a plurality of stimulation interference signals 409A-409N (collectively “stimulation interference signals 409”). In the example of FIG. 4A, control pulses 404 may also provide therapy to the patient, and informed pulses may not be necessary for therapy.

First channel 402 is a time/voltage (and/or current) graph indicating the voltage (or current) of at least one electrode of electrodes 232, 234. In one example, the stimulation electrodes of first channel 402 may be located on the opposite side of the lead as the sensing electrodes of second channel 406. Control pulses 404 may be electrical pulses delivered to the spinal cord of the patient by at least one of electrodes 232, 234, and control pulses 404 may be balanced biphasic square pulses with an interphase interval. In other words, each of control pulses 404 are shown with a negative phase and a positive phase separated by an interphase interval. For example, a control pulse 404 may have a negative voltage for the same amount of time and amplitude that it has a positive voltage. It is noted that the negative voltage phase may be before or after the positive voltage phase. Control pulses 404 may be delivered according to ECAP test stimulation programs 218 stored in storage device 212 of IMD 200, and ECAP test stimulation programs 218 may be updated according to user input via an external programmer and/or may be updated according to a signal from sensor(s) 222. In one example, control pulses 404 may have a pulse width of less than approximately 170 microseconds (e.g., the total time of the positive phase, the negative phase, and the interphase interval is less than 300 microseconds). In another example, control pulses 404 may have a pulse width of approximately 100 μs for each phase of the bi-phasic pulse. However, control pulses 404 may have longer pulses widths in other examples. As illustrated in FIG. 4A, control pulses 404 may be delivered via one or more electrodes that deliver or sense signals corresponding to channel 402. Delivery of control pulses 404 may be delivered by leads 230 in a guarded cathode electrode combination. For example, if leads 230 are linear 8-electrode leads, a guarded cathode combination is a central cathodic electrode with anodic electrodes immediately adjacent to the cathodic electrode. For some patients, control pulses 404 may sufficiently provide therapy that treats the condition and/or symptoms of the patient. Therefore, additional informed pulses may not be needed for these patients or for at least some aspect of the therapy for these patients.

Second channel 406 is a time/voltage (and/or current) graph indicating the voltage (or current) of at least one electrode of electrodes 232, 234. In one example, the electrodes of second channel 406 may be located on the opposite side of the lead as the electrodes of first channel 402. ECAPs 408 may be sensed at electrodes 232, 234 from the spinal cord of the patient in response to control pulses 404. ECAPs 408 are electrical signals which may propagate along a nerve away from the origination of control pulses 404. In one example, ECAPs 408 are sensed by different electrodes than the electrodes used to deliver control pulses 404. As illustrated in FIG. 4A, ECAPs 408 may be recorded on second channel 406.

Stimulation interference signals 409A, 409B, and 409N (e.g., the artifact of the stimulation pulses) may be sensed by leads 230 and may be sensed during the same period of time as the delivery of control pulses 404. Since the interference signals may have a greater amplitude and intensity than ECAPs 408, any ECAPs arriving at IMD 200 during the occurrence of stimulation interference signals 409 may not be adequately sensed by sensing circuitry 206 of IMD 200. However, ECAPs 408 may be sufficiently sensed by sensing circuitry 206 because each ECAP 408 falls after the completion of each a control pulse 404. As illustrated in FIG. 4A, stimulation interference signals 409 and ECAPs 408 may be recorded on channel 406.

FIG. 4B is a timing diagram 400B illustrating one example of electrical stimulation pulses and respective sensed ECAPs according to some techniques of the disclosure. For convenience, FIG. 4B is described with reference to IMD 200 of FIG. 2A. As illustrated, timing diagram 400B includes first channel 410, a plurality of control pulses 412A-412N (collectively “control pulses 412”), second channel 420, a plurality of informed pulses 424A-424N (collectively “informed pulses 424”) including passive recharge phases 426A-426N (collectively “passive recharge phases 426”), third channel 430, a plurality of respective ECAPs 436A-436N (collectively “ECAPs 436”), and a plurality of stimulation interference signals 438A-438N (collectively “stimulation interference signals 438”).

First channel 410 is a time/voltage (and/or current) graph indicating the voltage (or current) of at least one electrode of electrodes 232, 234. In one example, the stimulation electrodes of first channel 410 may be located on the opposite side of the lead as the sensing electrodes of third channel 430. Control pulses 412 may be electrical pulses delivered to the spinal cord of the patient by at least one of electrodes 232, 234, and control pulses 412 may be balanced biphasic square pulses with an interphase interval. In other words, each of control pulses 412 are shown with a negative phase and a positive phase separated by an interphase interval. For example, a control pulse 412 may have a negative voltage for the same amount of time and amplitude that it has a positive voltage. It is noted that the negative voltage phase may be before or after the positive voltage phase. Control pulses 412 may be delivered according to ECAP test stimulation programs 218 stored in memory 250 of IMD 200, and ECAP test stimulation programs 218 may be updated according to user input via an external programmer and/or may be updated according to a signal from sensor 216. In one example, control pulses 412 may have a pulse width of less than approximately 300 microseconds (e.g., the total time of the positive phase, the negative phase, and the interphase interval is less than 300 microseconds). In another example, control pulses 412 may have a pulse width of approximately 100 μs for each phase of the bi-phasic pulse. As illustrated in FIG. 4B, control pulses 412 may be delivered via one or more electrodes that deliver or sense signals corresponding to channel 410. Delivery of control pulses 412 may be delivered by leads 230 in a guarded cathode electrode combination. For example, if leads 230 are linear 8-electrode leads, a guarded cathode combination is a central cathodic electrode with anodic electrodes immediately adjacent to the cathodic electrode.

Second channel 420 is a time/voltage (and/or current) graph indicating the voltage (or current) of at least one electrode of electrodes 232, 234 for the informed pulses. In one example, the electrodes of second channel 420 may partially or fully share common electrodes with the electrodes of first channel 410 and third channel 430. Informed pulses 424 may also be delivered by the same leads 230 that are configured to deliver control pulses 412. Informed pulses 424 may be interleaved with control pulses 412, such that the two types of pulses are not delivered during overlapping periods of time. However, informed pulses 424 may or may not be delivered by exactly the same electrodes that deliver control pulses 412. Informed pulses 424 may be monophasic pulses with pulse widths of greater than approximately 100 μs and less than approximately 1000 μs. Informed pulses 424 may be configured to have longer pulse widths than control pulses 412, but may be the same or shorter in other examples. As illustrated in FIG. 4B, informed pulses 424 may be delivered on channel 420. As described above, in some examples (not shown in FIG. 4B), each informed pulse 424 may be replaced by a plurality (or a “burst”) of consecutive informed pulses delivered between a pair of consecutive control pulses 412 (e.g., between control pulses 412A, 412B). In this manner, the control pulses 412 may be described as being partially interleaved with informed pulses 424.

Informed pulses 424 may be configured for passive recharge. For example, each informed pulse 424 may be followed by a passive recharge phase 426 to equalize charge on the stimulation electrodes. Unlike a pulse configured for active recharge, wherein remaining charge on the tissue following a stimulation pulse is instantly removed from the tissue by an opposite applied charge, passive recharge allows tissue to naturally discharge to some reference voltage (e.g., ground or a rail voltage) following the termination of the informed pulse. In some examples, the electrodes of the medical device may be grounded at the medical device body. In this case, following the termination of informed pulse 424, the charge on the tissue surrounding the electrodes may dissipate to the medical device, creating a rapid decay of the remaining charge at the tissue following the termination of the pulse. This rapid decay is illustrated in passive recharge phases 426. Passive recharge phase 426 may have a duration in addition to the pulse width of the preceding informed pulse 424. In other examples (not pictured in FIG. 4B), informed pulses 424 may be bi-phasic pulses having a positive and negative phase (and, in some examples, an interphase interval between each phase) which may be referred to as pulses including active recharge. An informed pulse that is a bi-phasic pulse may or may not have a following passive recharge phase. Informed pulses 424 may be defined, and be a part of, one or more stimulation programs. Although each of informed pulses 424 are illustrated as having the same parameter values (e.g., the same pulse width, amplitude, and pulse shape), some of informed pulses 424 may have one or more parameters that have different values from each other.

Third channel 430 is a time/voltage (and/or current) graph indicating the voltage (or current) of at least one electrode of electrodes 232, 234. In one example, the electrodes of third channel 430 may be located on the opposite side of the lead (or another lead) as the electrodes of first channel 410. ECAPs 436 may be sensed at electrodes 232, 234 from the spinal cord of the patient in response to control pulses 412. ECAPs 436 are electrical signals which may propagate along a nerve away from the origination of control pulses 412. In one example, ECAPs 436 are sensed by different electrodes than the electrodes used to deliver control pulses 412. As illustrated in FIG. 4B, ECAPs 436 may be recorded on third channel 430.

Stimulation interference signals 438A, 438B, and 438N (e.g., the artifact of the stimulation pulses) may be sensed by leads 230 and may be sensed during the same period of time as the delivery of control pulses 412 and informed pulses 424. Since the interference signals may have a greater amplitude and intensity than ECAPs 436, any ECAPs arriving at IMD 200 during the occurrence of stimulation interference signals 438 may not be adequately sensed by sensing circuitry 212 of IMD 200. However, ECAPs 436 may be sufficiently sensed by sensing circuitry 212 because each ECAP 436 falls after the completion of each a control pulse 412 and before the delivery of the next informed pulse 424. As illustrated in FIG. 4B, stimulation interference signals 438 and ECAPs 436 may be recorded on channel 430. In other examples, control pulses 412 and informed pulses 424 may have similar pulse widths, but vary in one or more other parameter values. If programmed trains of pulses 424 interfere with a sensing window for sensing one or more of ECAPs 436, the system may remove or move one or more pulses 424 in order to reduce the likelihood that an artifact from a pulse, or ECAP from a pulse, obscures ECAPs 426 elicited by respective pulses 412.

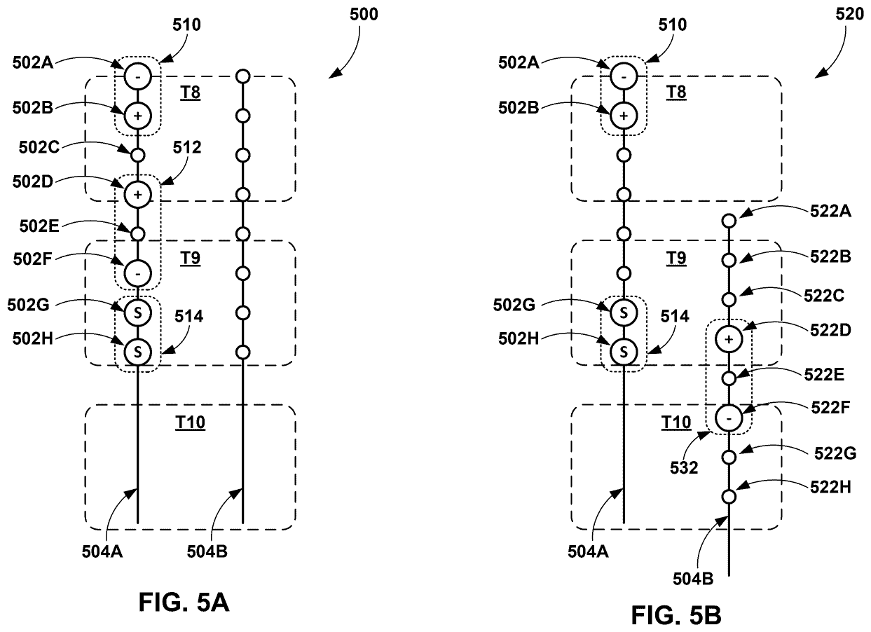

FIGS. 5A and 5B are conceptual diagrams of example leads and electrodes for delivering electrical stimulation and sensing ECAP signals. As shown in FIG. 5A, system 500 may include multiple leads 504A and 504B (collectively “leads 504”) that can be implanted within a patient. Leads 504 may be similar to leads 130 of FIG. 1 or leads 230 of FIG. 2A. Leads 504 are shown as they may be positioned with respect to different vertebra surround a spinal cord, such as vertebra T8, T9, and T10. However, leads 504 may have other positions in other examples. Leads 504 are shown as positioned at about the same vertebral levels, but could be offset in other examples such as shown in FIG. 5B. Lead 504A includes 8 electrodes 502A-502H (collectively “electrodes 502”), and lead 504B also includes 8 electrodes 522A-522H (collectively “electrodes 522”). However, other examples of system 500 may include leads with fewer, greater, or unequal numbers of electrodes. In addition, one, two, or three or more leads may be used in other examples.

In the example of FIG. 5A, stimulation pulses are delivered from different stimulation electrode combinations and a sensing electrode combination senses electrical signals all from the same lead 504A. A first program may define stimulation pulses delivered from stimulation electrode combination 512 that utilizes electrodes 502D and 502F. In some examples, stimulation electrode combination 512 may be used to deliver informed pulses that may be configured to provide therapy to the patient. A second program may define stimulation pulses delivered from stimulation electrode combination 510 that utilizes electrodes 502A and 502B. Stimulation electrode combination 510 may be used to deliver control pulses that may be configured to elicit evoked signals, such as ECAP signals. System 500 may be configured to sense ECAP signals elicited by stimulation electrode combination 510 using sensing electrode combination 514 that utilizes electrodes 502G and 502H. Generally, sensing electrode combination 514 may be set at the opposite end of stimulation electrode combination 510 in order to reduce the likelihood of artifacts from the pulses delivered by stimulation electrode combination 510 obscuring ECAP signals sensed by sensing electrode combination 514. Since all electrode combinations 510, 512, and 514 are on the same lead 504A, the timing of artifacts and ECAP signals may be easier to predict and more stable over time due to known distances between electrodes on lead 504A. However, it may still be advantageous to identify any artifacts within a sensing window to improve sensing performance of ECAP signals on sensing electrode combination 514.

FIG. 5B illustrates system 520 in which stimulation electrode combinations on different leads 504, which can result in unknown distances between stimulation and sensing electrode combinations. This difference in distances can further complicate accurate prediction of artifacts in sensing windows which could benefit from modifying stimulus schedules as described herein. In the example of FIG. 5B, stimulation pulses are delivered from different stimulation electrode combinations and a sensing electrode combination senses electrical signals all from different leads 504. A first program may define stimulation pulses delivered from stimulation electrode combination 532 that utilizes electrodes 522D and 522F. In some examples, stimulation electrode combination 532 may be used to deliver informed pulses that may be configured to provide therapy to the patient. Similar to FIG. 5A, a second program may define stimulation pulses delivered from stimulation electrode combination 510 that utilizes electrodes 502A and 502B. Stimulation electrode combination 510 may be used to deliver control pulses that may be configured to elicit evoked signals, such as ECAP signals. System 500 may be configured to sense ECAP signals elicited by stimulation electrode combination 510 using sensing electrode combination 514 that utilizes electrodes 502G and 502H. Although the same electrode combinations 510 and 514 are shown in FIG. 5B, other electrode combinations may be used in other examples.

Since stimulation electrode combination 532 is on lead 504B which is different than lead 504A. Due to this configuration, stimulus pulses delivered from stimulation electrode combination 532 could cause artifacts obscuring ECAP signals within the electrical signals sensed by sensing electrode combination 514. The distance between leads 504 may result in unknown timing from the delivery of pulses from stimulation electrode combination 532 to detection at sensing electrode combination 514. In addition, stimulus pulses from stimulation electrode combination 532 may elicit ECAP signals that could be detected at unknown times by sensing electrode combination 514. Therefore, the system may attempt to identify any artifacts within a sensing window configured to detect ECAPs elicited by pulse delivered from stimulation electrode combination 510, for example, and determine or adjust a stimulus schedule for delivery of pulses by one or more stimulation electrode combinations to reduce or eliminate artifacts from the sensing window. Such a stimulus schedule may improve detection of target ECAPs and therapeutic outcomes for the patient.

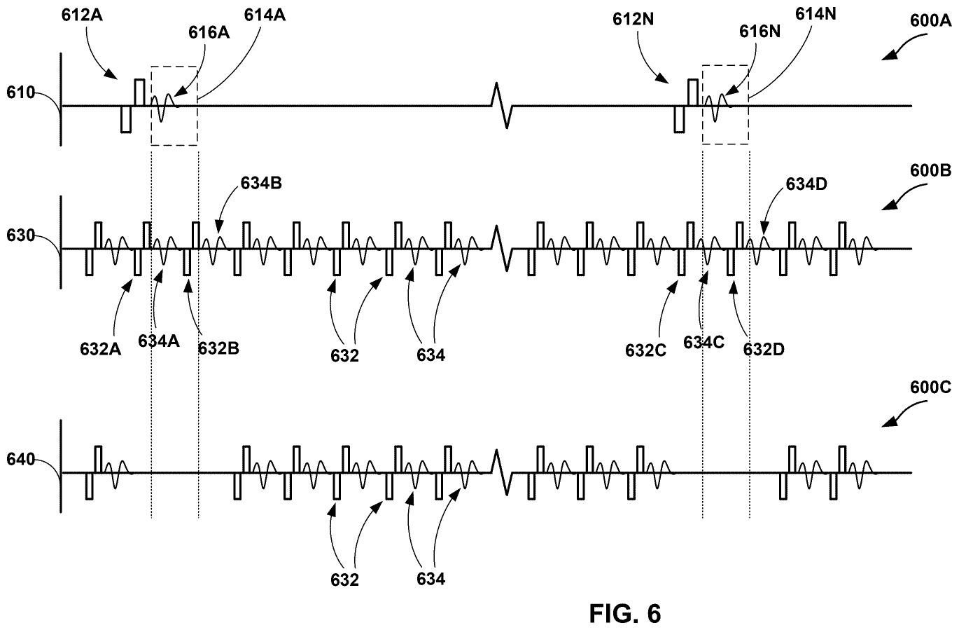

FIG. 6 is a timing diagram illustrating example blanking of pulses to generate a schedule for stimulation pulses. The delivery of pulses in timing diagrams 600A, 600B, and 600C are described with respect to IMD 200, but any medical device may be configured to deliver these pulses and sense electrical signals. As shown in the example of FIG. 6, timing diagram 600A indicates the timing of delivered stimulation pulses 612A and 612N (collectively “pulses 612”) and resulting ECAP signals 616A and 616N (collectively “ECAPs 616”) for a control program, such as control pulses delivered from one stimulation electrode combination. Stimulation electrode combination 510 of FIGS. 5A and 5B may deliver pulses 612, for example. Timing diagram 600B indicates the timing of delivered stimulation pulses 632 and resulting ECAP signals 634 that may occur for an informed program, such as informed pulses delivered from another stimulation electrode combination. Stimulation electrode combinations 512 of 532 of FIGS. 5A and 5B may deliver pulses 632, for example.

The pulse trains provided in timing diagrams 600A and 600B may be referred to a schedule of pulses or stimulus schedule. These pulses may be defined to be delivered at a predetermined pulse rate or some other predefined schedule. In some examples, each pulse train may have its own schedule, or single stimulus schedule may define the timing of delivery of pulses from multiple electrode combinations (such as both timing diagrams 600A and 600B).

As shown in timing diagram 600A, sensing windows 614A and 614N (collectively “sensing windows 614”) may be predetermined in time for when sensing circuitry 212 is scheduled to sense, or record, electrical signals via a sensing electrode combination, such as sensing electrode combination 514 of FIGS. 5A and 5B. Sensing windows 614 may be defined to occur at a time in which respective ECAP signals 616 are expected to occur after delivery of respective pulses 612. However, the delivery of pulses 632 may result in one or more pulses from that train of pulses being delivered during one or more sensing windows 614. In addition, or alternatively, one or more ECAPs 634 elicited by respective pulses 632 may occur within a respective sensing window 616. These pulses and/or unwanted ECAPs may be referred to as artifacts within the sensed electrical signal because they are unwanted signals different than the target evoked signals, such as ECAPs 616. In the example of FIG. 6, the pulse rate of pulses 612 may be 50 Hz, but the pulse rate may have a range from 40 Hz through 120 Hz in some examples. Example pulse widths may be 120 μs, 150 μs, or 200 μs. The pulse rate of pulses 632 may have a pulse rate of 1200 Hz or 900 Hz, and a range of pulse rates from 150 Hz through 1200 Hz may be used. In other examples, pulses 632 may be delivered in groups of pulses instead of continuous pulses. Example pulse widths may be 150 μs, 170 μs, or 200 μs for pulses 632. Each group of pulses may be initially scheduled to provide a blanking period for sensing windows 616, but that blanking period may not be accurate depending on the spatial arrangement of sensing and stimulation electrodes.

IMD 200, or another device, may alter the stimulus schedule of one or more pulse trains, such as pulses 634 of timing diagram 600B, in order to reduce the impact of these artifacts from obscuring the desired signal during sensing windows 614. For example, IMD 200 or another device may analyze the electrical signals sensed during a sensing window, such as sensing window 614A. IMD 200 can then identify any artifacts that may have occurred during sensing window 614A. IMD 200 may identify artifacts using various signal processing techniques such an identifying peaks over an amplitude threshold, certain frequencies above a power threshold during a spectral analysis, or any other techniques. Once IMD 200 identifies an artifact, IMD 200 can identify the pulse, or pulses, that were causing the artifacts and remove them from subsequent pulse trains.

As shown in FIG. 6, IMD 200 can identify that pulse 632B occurred during sensing window 616A and remove that pulse from pulses 632. Although pulse 632A was not identified within sensing window 616A, the resulting ECAP 634A was identified in sensing window 616A. Therefore, IMD 200 may remove the preceding pulse 632A from pulses 632 in order to prevent ECAP 634A from being detected during another sensing window. A similar process can occur with respect to sensing window 616N, where IMD 200 can remove pulses 632C and 632D such that those pulses and resulting ECAPs 634C and 634D do not cause artifacts within the electrical signals sensed during sensing window 614N. After removing these pulses, IMD 200 can generate a new, or adjusted, stimulus schedule shown as timing diagram 600C. As can be seen in timing diagram 600C, pulses 632A, 632B, 632C, and 632D have been removed from the pulse train that was previously scheduled to be delivered. In other examples, the initial timing diagram 600B may have groups, or bursts, of pulses initially scheduled to avoid sensing windows 616. However, due to locations of actual electrodes in tissue, IMD 200 may be able to move pulses in time and/or remove one or more pulses from these groups of pulses to avoid artifacts within sensing windows 616. In some examples, IMD 200 may be configured to add new pulses 632 to one or more groups of pulses where the new pulses still do not cause artifacts within any sensing windows. In any case, IMD 200 may use timing diagrams 600A and 600C after the analysis for any subsequent stimulation delivery. IMD 200 may perform this analysis initially during programming of therapy for the patient or rerun this analysis periodically or on demand at any time during use of IMD 200.

IMD 200 may perform this analysis of artifact identification and pulse removal/moving using various techniques. For example, pulse trains of pulses 612 and 632 may be aligned in time such that they have repeating segments of pulses over time. In this situation, IMD 200 may deliver pulses 612 and 632 for the period of time that covers the repeatable sequence. This sequency may include one sensing window 614 or multiple different sensing windows 614. IMD 200 may identify any artifacts within each of the sensing windows and remove, or add, pulses as needed in order to establish “clean” sensing windows with no, or minimal, artifacts from pulses 632. IMD 200 can then determine the stimulus schedule for subsequent stimulation according to this repeatable pattern. In other examples, IMD 200 may identify intervals of time from the delivery of a pulse 612 needed for a corresponding sensing window to be free of artifacts from any of pulses 632. For example, IMD 200 may identify artifacts and then back calculate the interval between pulse 612 and when the artifact occurred and the timing of corresponding pulses 632 that may be associated with the artifact. IMD 200 can then establish a “blanking period” or some other interval from delivery of each pulse 612 during which pulses 632 should be withheld from delivery. In this example, the stimulus schedule for pulses 632 may be dynamic in that IMD 200 may continually calculate when to deliver the next pulses 632 based on delivery of pulses 612 and the following needed sensing window. This type of stimulus schedule may still be based on the artifact identification from a prior pulse train for pulses 632.

In some examples, IMD 200 may determine the stimulus schedule only after two or more iterations of removing, moving, or adding pulses from pulses 632 to remove artifacts from sensing windows 614. For example, IMD 200 may perform a first analysis and remove pulses as determined to create a new stimulus schedule. IMD 200 may then deliver pulses again using the new stimulus schedule and perform another analysis on the sensed electrical signals during one or more sensing windows 614. IMD 200 may, if any further artifacts are identified, remove one or more additional pulses. In addition, IMD 200 may add back one or more pulses as appropriate (e.g., if the wrong pulses were removed as a result of the previous analysis). IMD 200 can then create another stimulus schedule and again re-deliver stimulation using the another stimulus schedule to further check for possible artifacts. IMD 200 may perform this process repeatedly, or on an iterative basis, until the sensed electrical signals are free, or relatively free, of artifacts and the stimulus schedule satisfies any set criteria. In some examples, IMD 200 may transmit the finalized stimulus schedule for presentation to a user for confirmation prior to beginning therapy with the finalized stimulus schedule. In this example, IMD 200 may initiate therapy using the finalized stimulus schedule in response to clinician approval.

As described herein, processing circuitry 214 can be configured to control stimulation generation circuitry 211 to deliver, at a repeating interleaved basis, a control pulse from a control stimulation electrode combination and subsequent pulses from the informed electrode combination according to any determined or updated stimulus schedule. As discussed above, processing circuitry 214 can be configured to determine the stimulus schedule by at least removing one or more pulses 632 from a preliminary schedule for a plurality of subsequent pulses to reduce one or more artifacts identified in an analysis of the electrical signal sensed during one or more sensing windows 616.

In this manner, IMD 200 may be configured to iteratively remove one or more pulses from the train of pulses 632 according to a preliminary schedule and analyze respective sensed subsequent electrical signals until a subsequent analysis of the respective sensed subsequent electrical signals indicates artifacts associated with the plurality of pulses are no longer identifiable in the sensing window. IMD 200 can then determine the stimulus schedule to include remaining pulses of the plurality of pulses according to the preliminary schedule. In some examples, IMD 200 can be configured to control the stimulation generation circuitry 211 to deliver subsequent pulses 632 from the stimulation electrode combination according to the stimulus schedule and control sensing circuitry 212 to sense electrical signals during another sensing window associated with delivery of a control pulse from the control stimulation electrode combination that is different than the informed stimulation electrode combination. IMD 200 can then generate another analysis of the sensed electrical signal during the sensing window, where the analysis is configured to identify one or more artifacts associated with one or more pulses of the subsequent pulses 632. Based on this another analysis that no artifacts associated with pulses 632, IMD 200 can responsively determine an adjusted stimulus schedule that includes an additional one or more pulses to the subsequent pulses without adding any artifacts associated with the adjusted stimulus schedule to subsequent sensed electrical signals. In this manner, IMD 200 can remove, add, or even move pulses to generate new, or adjusted, stimulus schedules as needed in order to reduce artifacts from sensed electrical signals. IMD 200 may attempt to maintain pulse rates and/or number of pulses in each group of pulses according to initial stimulation programs when determining a stimulus schedule. In some examples, IMD 200 may update one or more stimulation programs as a result of the determined stimulus schedule.

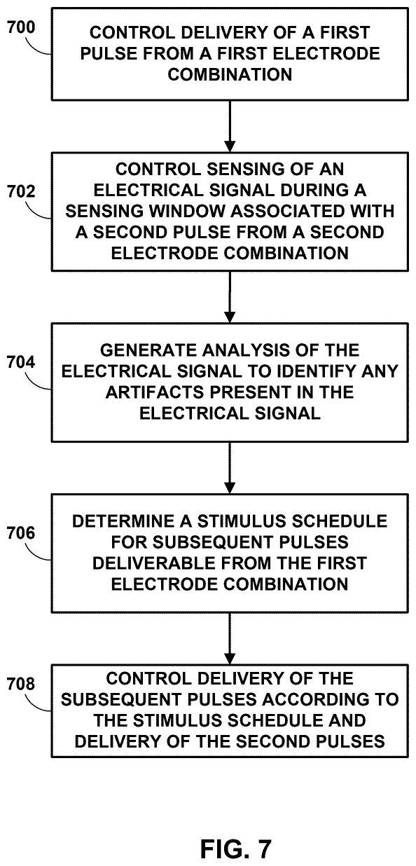

FIG. 7 is a flowchart illustrating an example technique for determining an stimulus schedule based on sensed signals from an electrode combination. For convenience, FIG. 7 is described with respect to IMD 200 of FIG. 2A. However, the techniques of FIG. 7 may be performed by different components of IMD 200 or by additional or alternative medical devices, such as programmer 150.

In the example of FIG. 7, processing circuitry 214 controls delivery of a first pulse (or first pulses) from a first electrode combination (700). This first electrode combination may be a stimulation electrode combination associated with informed pulses that are intended to deliver stimulation therapy to a patient. Processing circuitry 214 can then control IMD 200 to sense an electrical signal during a sensing window associated with a second pulse from a second electrode combination (702). This second pulse may be a control stimulation pulse from which IMD 200 is configured to attempt to detect an elicited ECAP signal.

Processing circuitry 214 can then generate, or perform, an analysis of the electrical signal that was sensed to identify any artifacts present in the electrical signal (704). For example, processing circuitry 214 may be configured to identify suprathreshold amplitudes indicative of sensed stimulation pulses, or that would not be representative of target ECAPs, partial ECAP signals, or unexpected frequencies of unexpected amplitude during a spectral analysis. Based on the identification of any artifacts, IMD 200 can determine a stimulus schedule for subsequent pulses deliverable from the first electrode combination (706). In some examples, IMD 200 may repeat steps 700-706 using the new stimulus schedule to confirm the schedule is correct or may make additional changes to the stimulus schedule. In some examples, IMD 200 may thus determine a pulse train or schedule for the pulses deliverable as informed stimulation pulses. IMD 200 can then control stimulation generator 211 to deliver subsequent pulses according to the stimulus schedule and delivery of the second pulses (708). The process of FIG. 7 can be performed during initial programming of therapy for the patient or performed at any time (e.g., periodically or in response to a user request or trigger event) to revisit sensing accuracy. ECAP signals are generally described as an example sensed electrical signal that can be used to identify any artifacts or other features for adjusting a stimulus schedule. However, other signals such as evoked resonant neural activity (ERNA) signals or other sensed signals, such as local field potential (LFP) signals, may be used instead of or in addition to ECAP signals. These other signals may be used in any other techniques described herein.

In some examples, the initial stimulus schedule (and/or timing the initial sensing window) may be predetermined, based on historical data, or even based on patient imaging (e.g., x-ray or fluoroscopy imaging) of electrodes. In one example, the system may measure distances between electrodes using the imaging data and estimate timing of stimulation based on the distances and estimated conduction velocities (e.g., average tissue conduction velocity or even tissue-specific conduction velocities). In this manner, the initial sensing windows from the stimulus schedule may be a closer match for the patient than starting with no initial distance information. In other examples, the initial stimulus schedule and/or sensing window may be based on an initial implantation plan by the clinician such that estimated electrode distances, and resulting stimulus schedule and/or sensing windows, are calculated based on that initial implantation plan or some post-operation modification thereof.

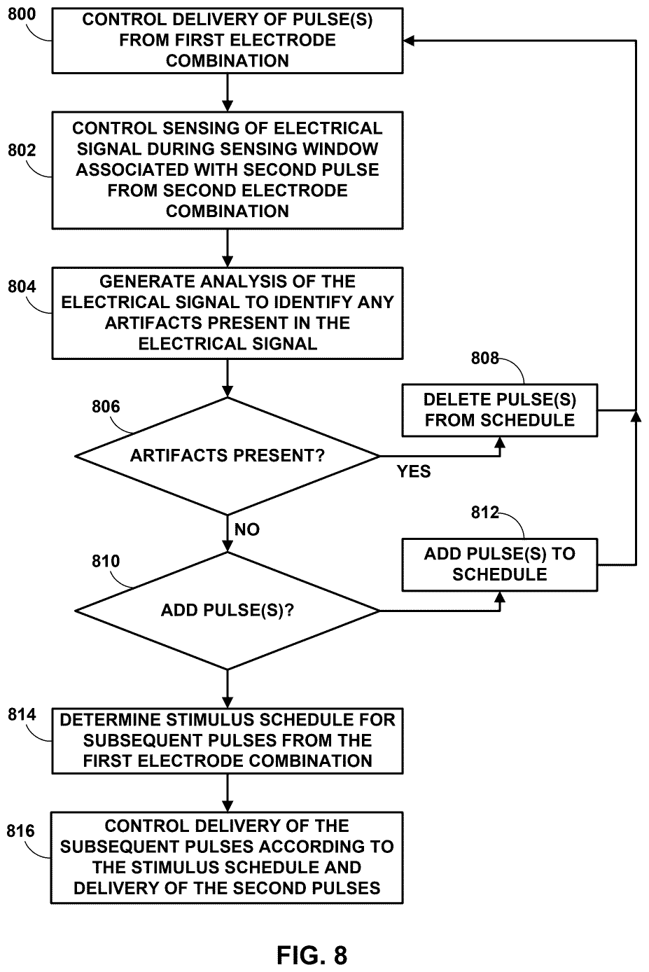

FIG. 8 is a flowchart illustrating an example technique for adding and removing pulses based on identified artifacts to determine a stimulus schedule for stimulation. For convenience, FIG. 8 is described with respect to IMD 200 of FIG. 2A. However, the techniques of FIG. 8 may be performed by different components of IMD 200 or by additional or alternative medical devices, such as programmer 150.

In the example of FIG. 8, processing circuitry 214 controls delivery of a first pulse (or first pulses) from a first electrode combination (800). This first electrode combination may be a stimulation electrode combination associated with informed pulses that are intended to deliver stimulation therapy to a patient. Processing circuitry 214 can then control IMD 200 to sense an electrical signal during a sensing window associated with a second pulse from a second electrode combination (802). This second pulse may be a control stimulation pulse from which IMD 200 is configured to attempt to detect an elicited ECAP signal.

Processing circuitry 214 can then generate, or perform, an analysis of the electrical signal that was sensed to identify any artifacts present in the electrical signal (804). For example, processing circuitry 214 may be configured to identify suprathreshold amplitudes indicative of sensed stimulation pulses, or that would not be representative of target ECAPs, partial ECAP signals, or unexpected frequencies of unexpected amplitude during a spectral analysis. If there are artifacts identified in the electrical signal (“YES” branch of block 806), IMD 200 can delete that one or more corresponding pulse from the schedule for the first pulses from the first electrode combination (808) and then repeat the process of delivering pulses again from the first electrode combination (800).

If there are no artifacts in the electrical signal (“NO” branch of block 806), processing circuitry 214 can determine whether any pulses should be added to the schedule (810). For example, processing circuitry 214 may determine that there is time for one or more additional pulses without impacting the sensing window, which may improve therapeutic outcomes for the patient. If there are pulses to be added (“YES” branch of block 810), IMD 200 can add that one or more pulse to the schedule for the first pulses from the first electrode combination (812) and then repeat the process of delivering pulses again from the first electrode combination (800).

If there are no pulses to be added (“NO” branch of block 810), processing circuitry 214 can then determine a stimulus schedule for subsequent pulses deliverable from the first electrode combination (814). In some examples, IMD 200 may thus determine a pulse train or schedule for the pulses deliverable as informed stimulation pulses. IMD 200 can then control stimulation generator 211 to deliver subsequent pulses according to the stimulus schedule and delivery of the second pulses (816). The process of FIG. 8 can be performed during initial programming of therapy for the patient or performed at any time (e.g., periodically or in response to a user request or trigger event) to revisit sensing accuracy.

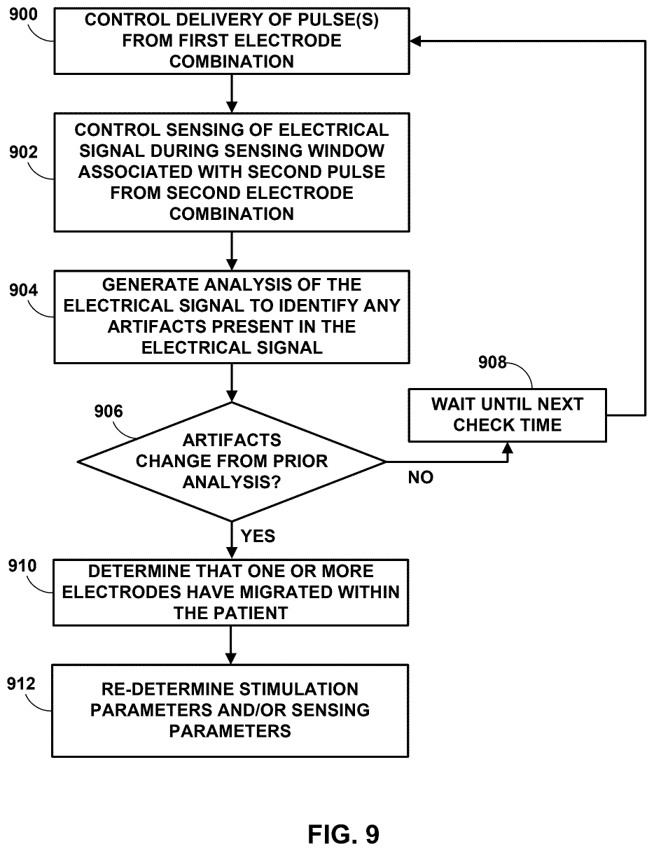

FIG. 9 is a flowchart illustrating an example technique for determining electrode migration based on a change in identified artifacts compared to a prior period of time. For convenience, FIG. 9 is described with respect to IMD 200 of FIG. 2A. However, the techniques of FIG. 9 may be performed by different components of IMD 200 or by additional or alternative medical devices, such as programmer 150.

In the example of FIG. 9, processing circuitry 214 controls delivery of a first pulse (or first pulses) from a first electrode combination (900). This first electrode combination may be a stimulation electrode combination associated with informed pulses that are intended to deliver stimulation therapy to a patient. Processing circuitry 214 can then control IMD 200 to sense an electrical signal during a sensing window associated with a second pulse from a second electrode combination (902). This second pulse may be a control stimulation pulse from which IMD 200 is configured to attempt to detect an elicited ECAP signal.

Processing circuitry 214 can then generate, or perform, an analysis of the electrical signal that was sensed to identify any artifacts present in the electrical signal (904). For example, processing circuitry 214 may be configured to identify suprathreshold amplitudes indicative of sensed stimulation pulses, or that would not be representative of target ECAPs, partial ECAP signals, or unexpected frequencies of unexpected amplitude during a spectral analysis. If there are no changes to the artifacts identified during a previous analysis (“NO” branch of block 906), processing circuitry 214 may wait a predetermined amount of time (908) before checking for artifacts and potential migration another time (900). Processing circuitry 214 may determine that there is a change in the artifacts if there are artifacts this time compared to no artifacts in the prior analysis. In some examples, prior analysis may still have had one or more artifacts. However, processing circuitry 214 can compare the artifacts for any change to the magnitude, timing, or any other aspects of the artifacts that may indicate electrode migration occurred.

If there are artifacts identified in the electrical signal that are different than the previous analysis (“YES” branch of block 906), processing circuitry 214 can determine that one or more electrodes have migrated within the patient (910). These electrodes may be electrodes part of the stimulation electrode combination and/or the sensing electrode combination. In some examples, processing circuitry 214 can infer distance changes between certain electrodes based on the timing of the artifact in the sensing window, such as artifacts moving later in time indicating electrodes moving further away and artifacts moving earlier in time moving closer together. In any case, electrode migration determination may trigger processing circuitry 214 to redetermine stimulation parameters and/or sensing parameters (912). This re-determination may include performing another analysis of artifacts in sensing windows in order to determine a new stimulus schedule such as in the techniques of FIG. 7 or 8. The process of FIG. 9 can be performed during initial programming of therapy for the patient or performed at any time (e.g., periodically or in response to a user request or trigger event) to revisit sensing accuracy.

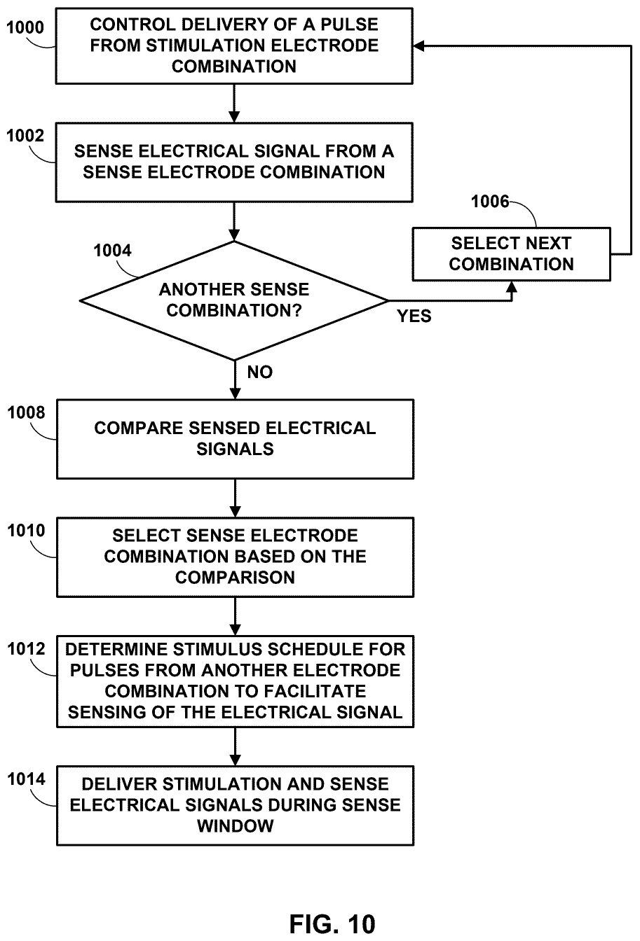

FIG. 10 is a flowchart illustrating an example technique for selecting a sense electrode combination and determining a stimulus schedule for adjusting stimulation therapy. For convenience, FIG. 10 is described with respect to IMD 200 of FIG. 2A. However, the techniques of FIG. 10 may be performed by different components of IMD 200 or by additional or alternative medical devices, such as programmer 150.

In the example of FIG. 10, processing circuitry 214 controls delivery of a pulse from a stimulation electrode combination (1000). The delivered pulse may be configured to elicit an evoked signal, such as an ECAP signal. The purpose of this process may be to evaluate potential sensing electrode combinations. Processing circuitry 214 than controls sensing circuitry 212 to sensing an electrical signal from a sensing electrode combination (1002). If there is another sensing electrode combination to try (“YES” branch of block 1004), processing circuitry 214 can select the next sensing electrode combination and again control delivery of a pulse from the stimulation electrode combination (1000) and sense with the new sensing electrode combination (1002). The different electrode combinations may include any combination of one or more adjacent electrode pairs, one or more wide sensing pairs (e.g., electrode pairs with one unused electrode between the pair), one or more double wide sensing pairs (e.g., electrode pairs with two unused electrodes between the pair), one or more electrode pairs on opposite ends of the same lead, one or more electrode pairs from different leads, one or more unipolar sensing pairs (e.g., one electrode is a ground at a housing of the IMD), or any other types of sensing electrode configurations. A sensing electrode combination may be on the same lead as the stimulation electrode combination or a different lead as the stimulation electrode combination.

If there are no further sensing electrode combinations to try (“NO” branch of block 1004), processing circuitry 214 can compare all of the sensed electrical signals from each of the respective sensing electrode combinations (1008). For example, processing circuitry 214 may apply thresholds and/or templates to each sensed electrical signal to identify ECAPs and/or artifacts within the sensed signals. In other examples, processing circuitry 214 may overlay the sensed signals on each other or otherwise identify various characteristics that can be compared, such as any artifact amplitudes, overlap from artifact to ECAP signal, amplitude of ECAP signal feature(s), etc. In one example, processing circuitry 214 may attempt to identify the sensing electrode configuration associated with the smallest artifact and largest ECAP amplitude.

Processing circuitry 214 can then select the sensing electrode combination based on the comparison (1010). In this manner, the sensing electrode combination may be selected according to patient-specific conditions such as tissue conductance and electrode locations within the patient. Processing circuitry 214 may then determine the stimulus schedule for stimulation therapy using the sensing electrode combination for pulses delivered from another stimulation electrode combination to facilitate sensing of the electrical signal and target ECAP or other evoked signal (1012). In some examples, the process of selecting the sensing electrode combination may combine the comparison of sensed signals to possible sensing windows. For example, processing circuitry 214 may use weighted criteria to optimize the sensing electrode combination with a sensing window that enables delivery of desired therapy pulses from another stimulation electrode combination with minimal additional artifacts. In this manner, one sensing electrode combination may have a less than preferred ECAP amplitude, but perhaps the available sensing window enables the system to deliver additional therapy pulses in the stimulus schedule. In any case, processing circuitry 214 can then control stimulation circuitry 211 to deliver subsequent stimulation and sense electrical signals during the determined sense window according to the determined stimulus schedule (1014). The process of FIG. 10 can be performed during initial programming of therapy for the patient or performed at any time (e.g., periodically or in response to a user request or trigger event) to revisit sensing accuracy.

The following examples are examples systems, devices, and methods described herein.

Example 1: A system comprising: processing circuitry configured to: control stimulation generation circuitry to deliver at least one first pulse from a first electrode combination; control sensing circuitry to sense an electrical signal during a sensing window associated with delivery of a second pulse from a second electrode combination different than the first electrode combination; generate an analysis of the electrical signal sensed during the sensing window, the analysis configured to identify one or more artifacts associated with the at least one first stimulus from the first electrode combination; and determine, based on the analysis, a stimulus schedule for a plurality of subsequent pulses deliverable from the first electrode combination.

Example 2. The system of example 1, wherein the processing circuitry is configured to control the stimulation generation circuitry to deliver, at a repeating interleaved basis, the second pulse from the second electrode combination and the plurality of subsequent pulses from the first electrode combination according to the stimulus schedule.

Example 3. The system of any of examples 1 or 2, wherein the one or more artifacts comprises at least one of a sensed signal from the first electrode combination or an evoked signal elicited by the at least one first pulse from the first electrode combination.

Example 4. The system of any of examples 1 through 3, wherein the sensing window is configured to enable sensing of an evoked compound action potential (ECAP) elicited by the second pulse from the second electrode combination.

Example 5. The system of any of examples 1 through 4, wherein the processing circuitry is configured to determine the stimulus schedule by at least removing one or more pulses from a preliminary schedule for the plurality of subsequent pulses to reduce the one or more artifacts identified in the analysis of the electrical signal sensed during the sensing window.

Example 6. The system of any of examples 1 through 5, wherein the at least one first pulse comprises a plurality of pulses from the first electrode combination according to a preliminary schedule, and wherein processing circuitry is configured to determine the stimulus schedule by at least: iteratively removing one or more pulses from the plurality of pulses according to the preliminary schedule and analyzing respective sensed subsequent electrical signals until a subsequent analysis of the respective sensed subsequent electrical signals indicates artifacts associated with the plurality of pulses are no longer identifiable in the sensing window; and determine the stimulus schedule to include remaining pulses of the plurality of pulses according to the preliminary schedule.

Example 7. The system of any of examples 1 through 6, wherein the processing circuitry is configured to: control the stimulation generation circuitry to deliver the plurality of subsequent pulses from the fist electrode combination according to the stimulus schedule; control the sensing circuitry to sense a second electrical signal during the sensing window associated with delivery of the second pulse from the second electrode combination different than the first electrode combination; generate a second analysis of the second electrical signal sensed during the sensing window, the analysis configured to identify one or more artifacts associated with one or more pulses of the plurality of subsequent pulses from the first electrode combination; determine, based on the second analysis, that no artifacts associated with the one or more pulses of the plurality of subsequent pulses from the first electrode combination are present in the second electrical signal; and determine an adjusted stimulus schedule that includes an additional one or more pulses to the subsequent pulses without adding any artifacts associated with the adjusted stimulus schedule to subsequent sensed electrical signals.

Example 8. The system of any of examples 1 through 7, wherein the electrical signal is a first electrical signal and the analysis is a first analysis, and wherein the processing circuitry is configured to: control the stimulation generation circuitry to deliver the plurality of subsequent pulses from the fist electrode combination according to the stimulus schedule; control the sensing circuitry to sense a second electrical signal during the sensing window associated with delivery of the second pulse from the second electrode combination different than the first electrode combination; generate a second analysis of the second electrical signal sensed during the sensing window, the analysis configured to identify one or more artifacts associated with one or more pulses of the plurality of subsequent pulses from the first electrode combination; and determine, based on the second analysis, that one or more electrodes of an electrode array has moved within a patient, wherein the electrode array comprises at least the first electrode combination or the second electrode combination.

Example 9. The system of any of examples 1 through 8, wherein the plurality of subsequent pulses have a first frequency in a first range from 150 Hz through 1200 Hz, and wherein the processing circuitry is configured to deliver the second pulse at a second frequency in a second range from 40 Hz through 120 Hz.

Example 10. The system of any of examples 1 through 9, wherein the sensing circuitry is configured to sense the electrical signal via a sensing electrode combination different from the first electrode combination and the second electrode combination.

Example 11. The system of any of examples 1 through 10, wherein the first electrode combination comprises at least one electrode disposed on a first lead, and wherein the second electrode combination comprises at least one electrode disposed on a second lead different from the first lead.

Example 12. The system of any of examples 1 through 11, further comprising an implantable medical device comprising the processing circuitry and the stimulation generation circuitry.

Example 13. A method comprising: controlling, by processing circuitry, stimulation generation circuitry to deliver at least one first pulse from a first electrode combination; controlling, by the processing circuitry, sensing circuitry to sense an electrical signal during a sensing window associated with delivery of a second pulse from a second electrode combination different than the first electrode combination; generating, by the processing circuitry, an analysis of the electrical signal sensed during the sensing window, the analysis configured to identify one or more artifacts associated with the at least one first stimulus from the first electrode combination; and determining, by the processing circuitry and based on the analysis, a stimulus schedule for a plurality of subsequent pulses deliverable from the first electrode combination.

Example 14. The method of example 13, further comprising controlling the stimulation generation circuitry to deliver, at a repeating interleaved basis, the second pulse from the second electrode combination and the plurality of subsequent pulses from the first electrode combination according to the stimulus schedule.

Example 15. The method of any of examples 13 or 14, wherein the one or more artifacts comprises at least one of a sensed signal from the first electrode combination or an evoked signal elicited by the at least one first pulse from the first electrode combination.

Example 16. The method of any of examples 13 through 15, wherein the sensing window is configured to enable sensing of an evoked compound action potential (ECAP) elicited by the second pulse from the second electrode combination.

Example 17. The method of any of examples 13 through 16, wherein determining the stimulus schedule further comprises removing one or more pulses from a preliminary schedule for the plurality of subsequent pulses to reduce the one or more artifacts identified in the analysis of the electrical signal sensed during the sensing window.

Example 18. The method of any of examples 13 through 17, wherein the at least one first pulse comprises a plurality of pulses from the first electrode combination according to a preliminary schedule, and wherein determining the stimulus schedule comprises: iteratively removing one or more pulses from the plurality of pulses according to the preliminary schedule and analyzing respective sensed subsequent electrical signals until a subsequent analysis of the respective sensed subsequent electrical signals indicates artifacts associated with the plurality of pulses are no longer identifiable in the sensing window; and determine the stimulus schedule to include remaining pulses of the plurality of pulses according to the preliminary schedule.

Example 19. The method of any of examples 13 through 18, wherein the electrical signal is a first electrical signal and the analysis is a first analysis, and wherein the method further comprises: controlling the stimulation generation circuitry to deliver the plurality of subsequent pulses from the fist electrode combination according to the stimulus schedule; controlling the sensing circuitry to sense a second electrical signal during the sensing window associated with delivery of the second pulse from the second electrode combination different than the first electrode combination; generating a second analysis of the second electrical signal sensed during the sensing window, the analysis configured to identify one or more artifacts associated with one or more pulses of the plurality of subsequent pulses from the first electrode combination; and determining, based on the second analysis, that one or more electrodes of an electrode array has moved within a patient, wherein the electrode array comprises at least the first electrode combination or the second electrode combination.

Example 20. A non-transitory computer-readable storage medium comprising instructions that, when executed, causes processing circuitry to: control stimulation generation circuitry to deliver at least one first pulse from a first electrode combination; control sensing circuitry to sense an electrical signal during a sensing window associated with delivery of a second pulse from a second electrode combination different than the first electrode combination; generate an analysis of the electrical signal sensed during the sensing window, the analysis configured to identify one or more artifacts associated with the at least one first stimulus from the first electrode combination; and determine, based on the analysis, a stimulus schedule for a plurality of subsequent pulses deliverable from the first electrode combination.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
processing circuitry configured to:
control stimulation generation circuitry to deliver at least one first pulse from a first electrode combination;
control sensing circuitry to sense an electrical signal during a sensing window associated with delivery of a second pulse from a second electrode combination different than the first electrode combination;
generate an analysis of the electrical signal sensed during the sensing window, the analysis configured to identify one or more artifacts associated with the at least one first stimulus from the first electrode combination; and
determine, based on the analysis, a stimulus schedule for a plurality of subsequent pulses deliverable from the first electrode combination.

2. The system of claim 1, wherein the processing circuitry is configured to control the stimulation generation circuitry to deliver, at a repeating interleaved basis, the second pulse from the second electrode combination and the plurality of subsequent pulses from the first electrode combination according to the stimulus schedule.

3. The system of claim 1, wherein the one or more artifacts comprises at least one of a sensed signal from the first electrode combination or an evoked signal elicited by the at least one first pulse from the first electrode combination.

4. The system of claim 1, wherein the sensing window is configured to enable sensing of an evoked compound action potential (ECAP) elicited by the second pulse from the second electrode combination.

5. The system of claim 1, wherein the processing circuitry is configured to determine the stimulus schedule by at least removing one or more pulses from a preliminary schedule for the plurality of subsequent pulses to reduce the one or more artifacts identified in the analysis of the electrical signal sensed during the sensing window.

6. The system of claim 1, wherein at least one first pulse comprises a plurality of pulses from the first electrode combination according to a preliminary schedule, and wherein processing circuitry is configured to determine the stimulus schedule by at least:
iteratively removing one or more pulses from the plurality of pulses according to the preliminary schedule and analyzing respective sensed subsequent electrical signals until a subsequent analysis of the respective sensed subsequent electrical signals indicates artifacts associated with the plurality of pulses are no longer identifiable in the sensing window; and
determine the stimulus schedule to include remaining pulses of the plurality of pulses according to the preliminary schedule.

7. The system of claim 1, wherein the processing circuitry is configured to:
control the stimulation generation circuitry to deliver the plurality of subsequent pulses from the fist electrode combination according to the stimulus schedule;
control the sensing circuitry to sense a second electrical signal during the sensing window associated with delivery of the second pulse from the second electrode combination different than the first electrode combination;
generate a second analysis of the second electrical signal sensed during the sensing window, the analysis configured to identify one or more artifacts associated with one or more pulses of the plurality of subsequent pulses from the first electrode combination;
determine, based on the second analysis, that no artifacts associated with one or more pulses of the plurality of subsequent pulses from the first electrode combination are present in the second electrical signal; and
determine an adjusted stimulus schedule that includes an additional one or more pulses to the subsequent pulses without adding any artifacts associated with the adjusted stimulus schedule to subsequent sensed electrical signals.

8. The system of claim 1, wherein the electrical signal is a first electrical signal and the analysis is a first analysis, and wherein the processing circuitry is configured to:
control the stimulation generation circuitry to deliver the plurality of subsequent pulses from the fist electrode combination according to the stimulus schedule;
control the sensing circuitry to sense a second electrical signal during the sensing window associated with delivery of the second pulse from the second electrode combination different than the first electrode combination;
generate a second analysis of the second electrical signal sensed during the sensing window, the analysis configured to identify one or more artifacts associated with one or more pulses of the plurality of subsequent pulses from the first electrode combination; and
determine, based on the second analysis, that one or more electrodes of an electrode array has moved within a patient, wherein the electrode array comprises at least the first electrode combination or the second electrode combination.

9. The system of claim 7, wherein the plurality of subsequent pulses have a first frequency in a first range from 150 Hz through 1200 Hz, and wherein the processing circuitry is configured to deliver the second pulse at a second frequency in a second range from 40 Hz through 120 Hz.

10. The system of claim 1, wherein the sensing circuitry is configured to sense the electrical signal via a sensing electrode combination different from the first electrode combination and the second electrode combination.

11. The system of claim 1, wherein the first electrode combination comprises at least one electrode disposed on a first lead, and wherein the second electrode combination comprises at least one electrode disposed on a second lead different from the first lead.

12. The system of claim 1, further comprising an implantable medical device comprising the processing circuitry and the stimulation generation circuitry.

13. A method comprising:
controlling, by processing circuitry, stimulation generation circuitry to deliver at least one first pulse from a first electrode combination;
controlling, by the processing circuitry, sensing circuitry to sense an electrical signal during a sensing window associated with delivery of a second pulse from a second electrode combination different than the first electrode combination;
generating, by the processing circuitry, an analysis of the electrical signal sensed during the sensing window, the analysis configured to identify one or more artifacts associated with the at least one first stimulus from the first electrode combination; and
determining, by the processing circuitry and based on the analysis, a stimulus schedule for a plurality of subsequent pulses deliverable from the first electrode combination.

14. The method of claim 13, further comprising controlling the stimulation generation circuitry to deliver, at a repeating interleaved basis, the second pulse from the second electrode combination and the plurality of subsequent pulses from the first electrode combination according to the stimulus schedule.

15. The method of claim 13, wherein one or more artifacts comprises at least one of a sensed signal from the first electrode combination or an evoked signal elicited by the at least one first pulse from the first electrode combination.

16. The method of claim 13, wherein the sensing window is configured to enable sensing of an evoked compound action potential (ECAP) elicited by the second pulse from the second electrode combination.

17. The method of claim 13, wherein determining the stimulus schedule further comprises removing one or more pulses from a preliminary schedule for the plurality of subsequent pulses to reduce the one or more artifacts identified in the analysis of the electrical signal sensed during the sensing window.

18. The method of claim 13, wherein at least one first pulse comprises a plurality of pulses from the first electrode combination according to a preliminary schedule, and wherein determining the stimulus schedule comprises:
iteratively removing one or more pulses from the plurality of pulses according to the preliminary schedule and analyzing respective sensed subsequent electrical signals until a subsequent analysis of the respective sensed subsequent electrical signals indicates artifacts associated with the plurality of pulses are no longer identifiable in the sensing window; and
determine the stimulus schedule to include remaining pulses of the plurality of pulses according to the preliminary schedule.

19. The method of claim 13, wherein the electrical signal is a first electrical signal and the analysis is a first analysis, and wherein the method further comprises:
controlling the stimulation generation circuitry to deliver the plurality of subsequent pulses from the fist electrode combination according to the stimulus schedule;
controlling the sensing circuitry to sense a second electrical signal during the sensing window associated with delivery of the second pulse from the second electrode combination different than the first electrode combination;
generating a second analysis of the second electrical signal sensed during the sensing window, the analysis configured to identify one or more artifacts associated with one or more pulses of the plurality of subsequent pulses from the first electrode combination; and
determining, based on the second analysis, that one or more electrodes of an electrode array has moved within a patient, wherein the electrode array comprises at least the first electrode combination or the second electrode combination.

20. A non-transitory computer-readable storage medium comprising instructions that, when executed, causes processing circuitry to:
control stimulation generation circuitry to deliver at least one first pulse from a first electrode combination;
control sensing circuitry to sense an electrical signal during a sensing window associated with delivery of a second pulse from a second electrode combination different than the first electrode combination;
generate an analysis of the electrical signal sensed during the sensing window, the analysis configured to identify one or more artifacts associated with the at least one first stimulus from the first electrode combination; and
determine, based on the analysis, a stimulus schedule for a plurality of subsequent pulses deliverable from the first electrode combination.
